(19) United States
(12) Patent Application Publication
(10) Pub. No.: US 2009/0062342 A1
(43) Pub. Date: Mar. 5, 2009

(54) AMINES

(75) Inventors: Olivier Bezencon, Riehen (CH); Daniel Bur, Therwil (CH); Olivier Corminboeuf, Allschwil (CH); Corinna Grisostomi, Allschwil (CH); Lubos Remen, Allschwil (CH); Sylvia Richard-Bildstein, Dietwiller (FR); Thomas Weller, Binningen (CH)

Correspondence Address: HOXIE & ASSOCIATES LLC, 75 MAIN STREET , SUITE 301, MILLBURN, NJ, 07041, US

(73) Assignee: Actelion Pharmaceuticals Ltd., Allschwil (CH)

(21) Appl. No.: 12/281,684

(22) PCT Filed: Mar. 7, 2007
(86) PCT No.: PCT/IB2007/050758

§ 371 Date: Sep. 4, 2008

(30) Foreign Application Priority Data
Mar. 8, 2006 (IB) 2006/050724

(51) Int. Cl. C07D 211/30; A61K 31/445; C07D 401/10; C07D 405/10; C07D 401/14; A61K 31/453; A61K 31/4545; A61P 9/00

(52) U.S. Cl. 514/318; 546/225; 514/330; 546/193; 546/207; 514/326

(57) ABSTRACT

The invention relates to novel amine derivatives and the use thereof as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel compounds of the formula (I). The invention also concerns related aspects including processes for the preparation of the compounds, pharmaceutical compositions containing one or more compounds of formula (I) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Birkenhager W. H., Reid J. L. (eds): Hypertension, Amsterdam, Elsevier Science Publishing Co, 1986, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). ACE inhibitors do not inhibit Chymase. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oefner C. et al., Chem. Biol., 1999, 6, 127; Patent Application WO 97/09311; Marki H. P. et al, Il Farmaco, 2001, 56, 21). However, the development status of these compounds is not known.

The present invention relates to renin inhibitors of a non-peptidic nature and of low molecular weight. Described are orally active renin inhibitors of formula (I) which have a long duration of action and which are active in indications beyond blood pressure regulation where the tissular renin-chymase system may be activated leading to pathophysiologically altered local functions such as renal, cardiac and vascular remodelling, atherosclerosis, and possibly restenosis. So, the present invention describes these non-peptidic renin inhibitors of formula (I).

In particular, the present invention relates to novel compounds of the formula (I)

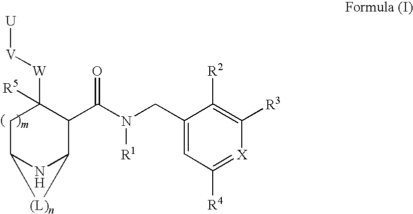

wherein
X represents CH, N, or N⁺—O—;
W represents a para-substituted phenyl, a para-substituted pyridinyl, or a thiazolyl, such as especially para-substituted phenyl or

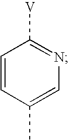

V represents —CH₂CH₂CH₂—, —CH₂CH₂-A-, —CH₂-A-CH₂—, -A-CH₂CH₂—, —CH₂CH₂CH₂CH₂—, -A-CH₂CH₂CH₂—, —CH₂-A-CH₂CH₂—, —CH₂CH₂-A-CH₂—, —CH₂CH₂CH₂-A-, -A-CH₂CH₂—B— (preferred), —CH₂CH₂CH₂CH₂CH₂—, -A-CH₂CH₂CH₂CH₂—, —CH₂-A-CH₂CH₂CH₂—, —CH₂CH₂-A-CH₂CH₂—, —CH₂CH₂CH₂-A-CH₂—, —CH₂CH₂CH₂CH₂-A-, -A-CH₂CH₂CH₂—B—, —CH₂-A-CH₂CH₂—B—, -A-CH₂CH₂—B—CH₂—, -A-CH₂CH₂CH₂—B—CH₂—, —CH₂-A-CH₂CH₂CH₂—B—, or —O—CH₂-Q- (also preferred), wherein Q is bound to the group U of formula (I), or (also preferably) V represents a pyrrolidinyl of the formula:

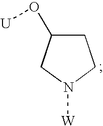

U represents unsubstituted aryl, especially phenyl; mono-, di-, tri- or tetra-substituted aryl (especially mono- di-, tri-, or tetra-substituted phenyl), wherein the substituents are independently selected from the group consisting of C₁₋₇-alkyl (such as especially methyl), —CF₃, halogen, and hydroxy-C₁₋₇-alkyl; or five-membered heteroaryl with two heteroatoms independently selected from nitrogen, oxygen and sulphur (preferably pyrazolyl or isoxazolyl), wherein said heteroaryl radical is optionally mono-, di- or tri-substituted, wherein the substitutents are independently selected from the group consisting of C₁₋₇-alkyl, C₁₋₇-alkoxy, —CF₃, —OCF₃, and halogen;
Q represents a five-membered heteroaryl with two or three heteroatoms independently selected from O and N, preferably an isoxazolyl, especially an isoxazolyl that is connected to the rest of the molecule of formula (I) as follows:

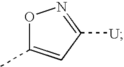

L represents —CH₂—CH₂—, —CH₂—CH(R⁶)—CH₂—, —CH₂—N(R⁷)—CH₂—, —CH₂—O—CH₂—, or —CH₂—S—CH₂—;
A and B represent independently from each others —O— or —S—;
R¹ represents C₁₋₇-alkyl or cycloalkyl, preferably cycloalkyl such as especially cyclopropyl;
R² represents halogen or C₁₋₇-alkyl, preferably chloro or methyl;
R³ represents hydrogen, halogen, C₁₋₇-alkyl (such as especially methyl), C₁₋₇-alkoxy, or —CF₃;
R⁴ represents hydrogen; C₁₋₁₇-alkyl-O—(CH₂)₀₋₄—CH₂—; CF₃—O—(CH₂)₀₋₄—CH₂—; R′₂N—(CH₂)₀₋₄—CH₂—, wherein R′ is independently selected from the group consisting of hydrogen, C₁₋₇-alkyl (optionally but preferably substituted by one to three fluorine), cyclopropyl (optionally substituted by one to three fluorine), cyclopropyl-C₁₋₇-alkyl (optionally but preferably substituted by one to three fluorine), and —C(═O)—R″ wherein R″ is C₁₋₄-alkyl, C₁₋₄-alkoxy, —CF₃, —CH₂—CF₃, or cyclopropyl; or R¹³—C(═O)—(O)O—, —(CH₂)₀₋₄—, wherein R¹³ is C₁₋₄-alkyl, C₁₋₄-alkoxy, or cyclopropyl; wherein R′ and R″ preferably do not both simultaneously represent hydrogen;
R⁵ represents hydroxy, C₁₋₇-alkoxy, hydroxy-C₁₋₇-alkyl, dihydroxy-C₁₋₇-alkyl, C₁₋₇-alkoxy-C₁₋₇-alkyl, C₁₋₇-alkoxy-C₁₋₇-alkoxy-C₁₋₇-alkyl, hydroxy-C₁₋₇-alkoxy-C₁₋₇-alkyl, carbamoyl-C₁₋₇-alkoxy, or C₁₋₇-alkyl-carbonyloxy;
R⁶ represents —H, —CH₂OR⁹, —CH₂NR⁸R⁹, —CH₂NR⁸COR⁹, —CH₂NR⁸SO₂R⁹, —CO₂R⁹, —CH₂OCONR⁸R⁹, —CONR⁸R⁹, —CH₂NR⁸CONR⁸′R⁹, —CH₂SO₂NR⁸R⁹, —CH₂SR⁹, —CH₂SOR⁹, or —CH₂SO₂R⁹;
R⁷ represents —R⁹, —COR⁹, —COOR¹¹, —CONR⁸R⁹, —C(NR⁸)NR⁸′R⁹, —CSNR⁸R⁹, —SO₂R⁹, or —SO₂NR⁸R⁹; or R⁷ represents a radical of the formula:

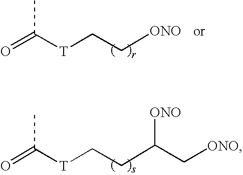

wherein T represents —CH₂—, —NH— or —O—, r is an integer from 1 to 6 and s is an integer from 1 to 4;
R⁸ and R⁸′ independently represent hydrogen, C₁₋₇-alkyl, C₂₋₇-alkenyl, cycloalkyl, or cycloalkyl-C₁₋₇-alkyl, wherein C₁₋₇-alkyl, cycloalkyl, and cycloalkyl-C₁₋₇-alkyl can be substituted by one, two, or three halogens;
R⁹ represents hydrogen, C₁₋₇-alkyl, cycloalkyl, or cycloalkyl-C₁₋₇-alkyl, wherein C₁₋₇-alkyl, cycloalkyl, and cycloalkyl-C₁₋₇-alkyl may be mono-, di- or tri-substituted, wherein the substituents are independently selected from the group consisting of halogen, hydroxy, —OCOR¹², —COOR¹², C₁₋₇-alkoxy, cyano, SO₂R¹², —CONR¹²R¹²′, morpholin-4-yl-CO, (4-C₁₋₇-alkyl)piperazin-1-yl)-CO—, —NHC(NH)NH₂, —NR¹⁰R¹⁰′ and C₁₋₇-alkyl, with the proviso that a carbon atom is attached at the most to one heteroatom in case this carbon atom is sp³-hybridized;
R¹⁰ and R¹⁰′ independently represent hydrogen, C₁₋₇-alkyl, cycloalkyl, cycloalkyl-C₁₋₇-alkyl, hydroxy-C₁₋₇-alkyl, —COOR⁸, or —CONH₂;
R¹¹ represents halogen, C₁₋₇-alkyl, C₁₋₇-alkoxy, —CF₃, or hydrogen;
R¹² and R¹²′ independently represent hydrogen, C₁₋₇-alkyl, C₂₋₇-alkenyl, cycloalkyl, or cycloalkyl-C₁₋₇-alkyl, wherein C₁₋₇-alkyl, cycloalkyl, and cycloalkyl-C₁₋₇-alkyl can be substituted by one, two, or three halogens;
n represents the integer 0 or 1, especially 0; and
m represents the integer 0 or 1, especially 1, with the proviso that m represents the integer 1 if n represents the integer 1;
and salts thereof.

The general terms used hereinbefore and hereinafter preferably have, within this disclosure, the following meanings, unless otherwise indicated:

Where the plural form is used for compounds, salts, pharmaceutical compositions, diseases and the like, this is intended to mean also a single compound, salt, or the like.

Any reference to a compound of formula (I) is to be understood as referring also to salts (especially pharmaceutically acceptable salts) of a compound of formula (I), as appropriate and expedient.

The term C₁₋₇-alkyl, alone or in combination with other groups, means saturated, straight or branched chain groups with one to seven carbon atoms, preferably one to four carbon atoms, i.e. C₁₋₄-alkyl. Examples of C₁₋₇-alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

The term C₁₋₇-alkoxy, alone or in combination with other groups, refers to an R—O— group, wherein R is a C₁₋₇-alkyl group. Examples of C₁₋₇-alkoxy groups are methoxy, ethoxy, propoxy, iso-propoxy, iso-butoxy, sec-butoxy and tert-butoxy.

The term hydroxy-C₁₋₇-alkyl, alone or in combination with other groups, refers to an HO—R group, wherein R is a C₁₋₇-alkyl group. Examples of hydroxy-C₁₋₇-alkyl groups are HO—CH₂—, HO—CH₂CH₂—, HO—CH₂CH₂CH₂— and CH₃CH(OH)—.

The term C₂₋₇-alkenyl, alone or in combination with other groups, means straight or branched chain groups comprising an olefinic bond and consisting of two to seven carbon atoms, preferably two to four carbon atoms. Examples of C₂₋₇-alkenyl are vinyl, propenyl and butenyl.

The term halogen means fluorine, chlorine, bromine or iodine, preferably fluorine, chlorine or bromine. In a more preferred embodiment of the invention the term halogen means fluorine or chlorine.

The term cycloalkyl, alone or in combination with other groups, means a saturated cyclic hydrocarbon ring system with 3 to 7 carbon atoms, i.e. cyclopropyl, cyclobutyl, cyclopentyl, cyclohexyl or cycloheptyl, preferably cyclopropyl.

The term aryl, alone or in combination, refers to a phenyl, naphthyl or indanyl group, preferably a phenyl group.

The term sp³-hybridized refers to a carbon atom and means that this carbon atom forms four bonds to four substituents placed in a tetragonal fashion around this carbon atom.

The expression pharmaceutically acceptable salts encompasses either salts with inorganic acids or organic acids like hydrochloric acid, hydrobromic acid, hydroiodic acid, sulfuric acid, sulfamic acid, phosphoric acid, nitric acid, phosphorous acid, nitrous acid, citric acid, formic acid, acetic acid, oxalic acid, maleic acid, lactic acid, tartaric acid, fumaric acid, benzoic acid, mandelic acid, cinnamic acid, palmoic acid, stearic acid, glutamic acid, aspartic acid, methanesulfonic acid, ethanesulfonic acid, ethanedisulfonic acid, p-toluenesulfonic acid, salicylic acid, succinic acid, trifluoroacetic acid, and the like that are non toxic to living organisms or in case the compound of formula (I) is acidic in nature with an inorganic base like an alkali or earth alkali base, e.g. sodium hydroxide, potassium hydroxide, calcium hydroxide and the like. For other examples of pharmaceutically acceptable salts, reference can be made to “Salt selection for basic drugs”, Int. J. Pharm. (1986), 33, 201-217.

The compounds of the formula (I) may contain asymmetric carbon atoms. Substituents at a double bond or a ring may be present in cis- (=Z-) or trans (=E-) form unless indicated otherwise. The compounds of formula (I) may thus be present as mixtures of stereoisomers or preferably as pure stereoisomers. Mixtures of stereoisomers may be separated in a manner known per se, e.g. by column chromatography, thin layer chromatography, HPLC or crystallization.

Compounds of the invention also include nitrosated compounds of formula (I) that have been nitrosated through one or more sites such as oxygen (hydroxyl condensation), sulfur (sulfydryl condensation) and/or nitrogen. The nitrosated compounds of the present invention can be prepared using conventional methods known to one skilled in the art. For example, known methods for nitrosating compounds are described in U.S. Pat. Nos. 5,380,758, 5,703,073, 5,994,294, 6,242,432 and 6,218,417; WO 98/19672; and Oae et al., Org. Prep. Proc. Int., 15(3): 165-198 (1983).

A preferred embodiment of the present invention relates to a compound of formula (I), wherein X represents N⁺—O⁻ and R⁴ represents C₁₋₄-alkoxy-C(═O)—NH—(CH₂)₀₋₄—CH₂— or R¹³—C(═O)—(O)₀₋₁—(CH₂)₀₋₄—, wherein R¹³ is C₁₋₄-alkyl, C₁₋₄-alkoxy, or cyclopropyl.

A preferred embodiment of the present invention relates to a compound of formula (I), wherein X represents CH or N; and

R⁴ represents hydrogen; C₁₋₇-alkyl-O—(CH₂)₀₋₄—CH₂—; CF₃—O—(CH₂)₀₋₄—CH₂—; or R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, —CF₃, —CH₂—CF₃, or cyclopropyl.

A preferred embodiment of the present invention relates to a compound of formula (I), wherein X represents CH or N⁺—O⁻.

A preferred embodiment of the present invention relates to a compound of formula (I), wherein R⁷ represents —R⁹, —COR⁹, —COOR¹¹, —CONR⁸R⁹, —C(NR⁸)NR⁸′R⁹, —CSNR⁸R⁹, —SO₂R⁹, or —SO₂NR⁸R⁹.

A preferred embodiment of the present invention relates to a compound of formula (I), wherein A and B both represent —O—.

A preferred embodiment of the present invention relates to a compound of formula (I), wherein R⁶ represents —CO₂CH₃ or —CO₂H.

A preferred embodiment of the present invention relates to a compound of formula (I), wherein R⁷ represents —H, —COCH₃, —C(NH)NH₂, —CONHCH₂C(CH₃)₂CONH₂, —CONHCH(CH₂)₂, or —CONHC(CH₂)₂CN.

A preferred embodiment of the present invention relates to a compound of formula (I), wherein R⁷ represents —H.

A preferred embodiment of the present invention relates to a compound of formula (I), wherein L represents —CH₂—CH₂— or —CH₂—NH—CH₂—.

A preferred embodiment of the present invention relates to a compound of formula (I), wherein R¹ represents cyclopropyl.

A preferred embodiment of the present invention relates to a compound of formula (I), wherein W represents a para-substituted phenyl, or

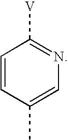

A preferred embodiment of the present invention relates to a compound of formula (I), wherein V represents —O—CH₂CH₂—O—, —O—CH₂-Q-, —CH₂—CH₂—O— wherein the —CH₂ part of —CH₂—CH₂—O— is bound to the group W of formula (I), or

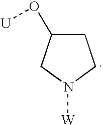

A preferred embodiment of the present invention relates to a compound of formula (I), wherein V represents —O—CH₂CH₂—O— or —O—CH₂-Q-.

A preferred embodiment of the present invention relates to a compound of formula (I), wherein Q represents an isoxazolyl or an oxadiazolyl.

A preferred embodiment of the present invention relates to a compound of formula (I), wherein Q represents an isoxazolyl, especially an isoxazolyl that is connected to the rest of the molecule of formula (I) as follows:

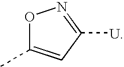

A preferred embodiment of the present invention relates to a compound of formula (I), wherein V-W represents:

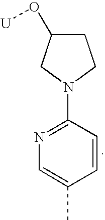

A preferred embodiment of the present invention relates to a compound of formula (I), wherein U represents:

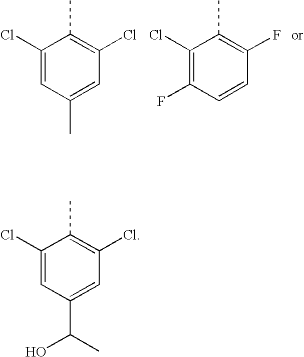

A preferred embodiment of the present invention relates to a compound of formula (I), wherein U represents:

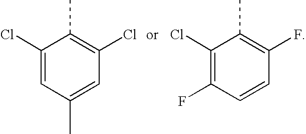

A preferred embodiment of the present invention relates to a compound of formula (I), wherein R² represents Cl, and R³ represents hydrogen.

A preferred embodiment of the present invention relates to a compound of formula (I), wherein R⁴ represents CH₃—O—(CH₂)₂₋₃— or CH₃—C(═O)—NH—CH₂—CH₂—.

A preferred embodiment of the present invention relates to a compound of formula (I), wherein R⁴ represents —CH₂CH₂CH₂—O—CH₃ or —CH₂CH₂—O—CH₃.

A preferred embodiment of the present invention relates to a compound of formula (I), wherein R⁴ represents —CH₂CH₂—O—CH₃.

A preferred embodiment of the present invention relates to a compound of formula (I), wherein R⁵ represents hydroxy.

A preferred embodiment of the present invention relates to a compound of formula (I), wherein n represents the integer 0.

A preferred embodiment of the present invention relates to a compound of formula (I), wherein the moiety

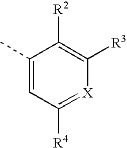

represents one of the following possibilities:

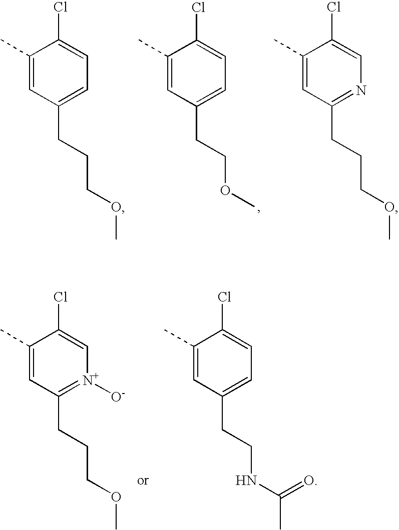

An especially preferred embodiment of the present invention relates to a compound of formula (I), wherein

X represents CH, N, or N⁺—O⁻;
W represents a para-substituted phenyl or a para-substituted pyridinyl, wherein the pyridinyl is especially connected to the rest of the molecule of formula (I) as follows:

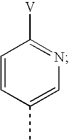

V represents -A-CH₂CH₂—B— or —O—CH₂-Q-, wherein Q is bound to the group U of formula (I), or V represents a pyrrolidinyl of the formula:

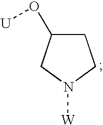

U represents tri-substituted phenyl, wherein the substituents are independently selected from the group consisting of C₁₋₇-alkyl (such as especially methyl) and halogen;
Q represents an isoxazolyl, especially an isoxazolyl that is connected to the rest of the molecule of formula (I) as follows:

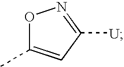

A and B both represent —O—;
R¹ represents cyclopropyl;
R² represents halogen or C₁₋₇-alkyl, especially chloro or methyl;
R³ represents hydrogen or C₁₋₇-alkyl, especially hydrogen or methyl;
R⁴ represents C₁₋₁₇-alkyl-O—(CH₂)₀₋₄—CH₂—, especially CH₃—O—(CH₂)₁₋₂—CH₂—;
R⁵ represents hydroxy;
n represents the integer 0; and
m represents the integer 1.

A preferred embodiment of the present invention relates to a compound of formula (I), wherein the absolute configuration of a compound of formula (I) is as represented for formula (I′):

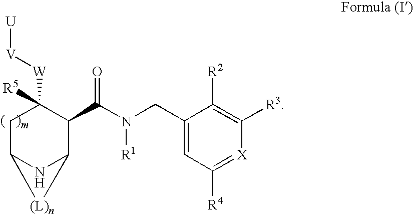

The present invention also relates to compounds of formula (I) wherein the meanings of one or more of the substituents and symbols as defined for formula (I), or a preferred embodiment of formula (I), are replaced by their preferred meanings as defined herein, such as those defined for the above-given preferred embodiments.

A preferred embodiment of the present invention relates to a compound of formula (I), which is (3S*,4R*)-4-{4-[2-(2,6-dichloro-4-methyl-phenoxy)-ethoxy]-phenyl}-4-hydroxy-piperidine-3-carboxylic acid cyclopropyl-(2,3-dimethyl-benzyl)-amide.

Another preferred embodiment of the present invention relates to a compound of formula (I) selected from:
(3S,4R)-4-{4-[2-(2,6-dichloro-4-methyl-phenoxy)-ethoxy]-phenyl}-4-hydroxy-piperidine-3-carboxylic acid [2-chloro-5-(2-methoxy-ethyl)-benzyl]-cyclopropyl-amide,
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[3-(2-chloro-3,6-difluoro-phenyl)-isoxazol-5-ylmethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[(R)-3-(2,6-dichloro-4-methyl-phenoxy)-pyrrolidin-1-yl]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-pyridin-4-ylmethyl]-cyclopropyl-amide, and
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-1-oxy-pyridin-4-ylmethyl]-cyclopropyl-amide.

The compounds of formula (I) are useful for the treatment and/or prophylaxis of diseases such as or related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases related to the renin-angiotensin system.

The compounds of formula (I) are especially useful for the treatment and/or prophylaxis of hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment and/or prophylaxis of diseases, which are associated with a dysregulation of the renin-angiotensin system, in particular to a method for the treatment and/or prophylaxis of the above-mentioned diseases, said methods comprising administering to a patient a pharmaceutically active amount of a compound of formula (I).

A further aspect of the present invention relates to pharmaceutical compositions comprising a compound of formula (I) and a pharmaceutically acceptable carrier material. These pharmaceutical compositions may be used for the treatment and/or prophylaxis of the above-mentioned diseases. The pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The invention also relates to the use of a compound of formula (I) for the preparation of pharmaceutical compositions for the treatment and/or prophylaxis of the above-mentioned diseases.

The production of the pharmaceutical compositions can be effected in a manner which will be familiar to any person skilled in the art (see for example Mark Gibson, Editor, Pharmaceutical Preformulation and Formulation, IHS Health Group, Englewood, Colo., USA, 2001; Remington, The Science and Practice of Pharmacy, 20th Edition, Philadelphia College of Pharmacy and Science) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Compounds of formula (I) or the above-mentioned pharmaceutical compositions are also of use in combination with other pharmacologically active compounds such as ACE-inhibitors, neutral endopeptidase inhibitors, aldosterone antagonists, angiotensin TI receptor antagonists, endothelin receptors antagonists, vasodilators, calcium antagonists, potassium activators, diuretics, sympatholitics, beta-adrenergic antagonists, alpha-adrenergic antagonists, 11beta-hydroxysteroid dehydrogenase type 1 inhibitors, soluble guanylate cyclase activators and/or other drugs beneficial for the prevention or the treatment of the above-mentioned diseases.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The compounds of formula (I) can be manufactured by the methods outlined below, by the methods described in the examples or by analogous methods.

A compound of type A (see patent applications WO 2003/093267, WO 2004/002957, WO 2004/096769, WO 2004/096803, WO 2004/096799, and WO 2004/096366) as described in Scheme 1 can be transformed into a compound of type B, wherein L′ stands for a precursor of the group L as defined for formula (I), and R^{a} for a typical ester substituent, like methyl, ethyl, or benzyl. PG stands for a suitable protecting group, typically a carbamate, a benzyl, or a methyl. Scheme 1 represents a compound of formula (I) wherein m is the integer 1; the same scheme can be used if m and n represent the integers 0, but m was omitted in the Scheme for the purpose of clarity. L′ can be modified along the synthesis. The amine has to be prepared separately (vide infra for specific examples). An alkylation of the ketone of a compound of type B leads to a compound of type C, or, if the U-V-W-segment is already achieved, to a compound of type D. V^{a} stands for a precursor of V as defined for formula (I), and can be transformed along the synthesis. Achievement of the U-V-W-segment in a compound of type C leads to a compound of type D. Alkylation or acylation of the tertiary alcohol in a compound of type D leads to a compound of type E. Final achievement of the L-substituent leads to a compound of type F. Deprotection will finally yield a compound of formula (I).

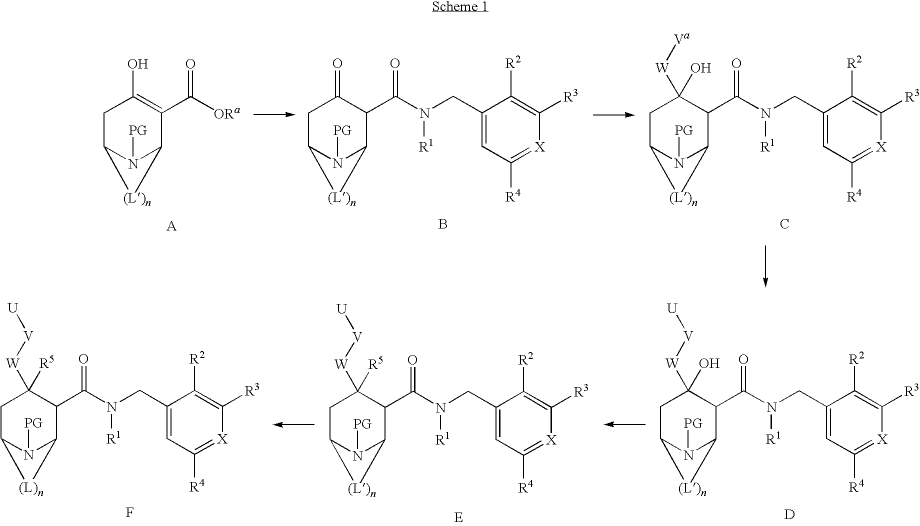

The alkylation of a compound of type B to a compound of type C yields a mixture of diastereoisomers. These diastereoisomers can be separated at this stage, or at any later stage (compounds of type D, E, F, or compound of formula (I)).

The preparation of several U-V-W- or V^{a}-W-substituents is described in the patent applications mentioned earlier. Otherwise a pyrrolidine substituent can be attached to an aromatic ring by a copper- or palladium-catalysed coupling as described in Scheme 2.

Under certain circumstances a transition metal is not necessary to catalyse this reaction. A compound of type G, wherein PG′ stands for a suitable protecting group, will be transformed into a compound of type H, wherein X′ stands for CH or N. If W in formula (I) represents a thiazolyl, the same chemistry can be applied as well.

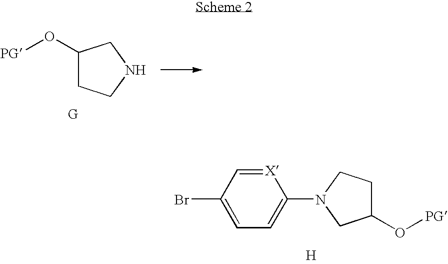

If V represents —O—CH₂-Q-, the isoxazolyl moiety is prepared by cycloaddition. This cycloaddition can be realized on the W-V^{a}-fragment in a compound of type C, leading to a compound of type D as described in Scheme 1. Otherwise the cycloaddition can be performed separately as, for instance, described in Scheme 3. Cycloaddition on a compound of type J with an often commercially available aldehyde leads to a compound of type K. Of course the aldehyde moiety can be built on the W-V^{a}-fragment, and a compound of the form U-CCH can be constructed, to give after cycloaddition another isoxazolyl moiety. The same principles can be used to prepare oxadiazolyl moieties, using methodologies described in the literature.

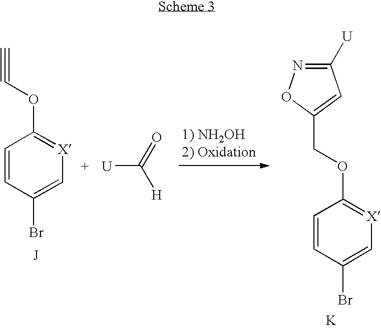

Also a hydroxymethyl isoxazole (Scheme 4) can be prepared from the aldehyde mentioned in Scheme 3 and propargyl alcohol. Coupling to a phenyl or heteroaryl derivative, wherein X″ typically stands for —OH, —Br, or —I, leads to a compound of type K.

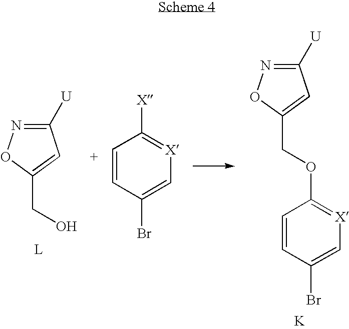

The following examples serve to illustrate the present invention in more details. They are, however, not intended to limit its scope in any manner.

### EXPERIMENTAL PART

Abbreviations (as used herein):
AcOH acetic acid
Ang angiotensin
aq. aqueous
Boc tert-butyloxycarbonyl
BSA bovine serum albumine
Bu butyl
BuLi n-butyllithium
Cy cyclohexyl
dba dibenzylidene acetone
DIPEA diisopropylethylamine

### DMAP 4-N,N-dimethylaminopyridine

### DMF N,N-dimethylformamide

DMPU 1,3-dimethyl-3,4,5,6-tetrahydro-2(1H)-pyrimidinone
DMSO dimethylsulfoxide
dppp 1,3-bis(diphenylphosphino)propane
EDC.HCl ethyl-N,N-dimethylaminopropylcarbodiimide hydrochloride
EIA enzyme immunoassay
ELSD evaporative light scattering detection
eq. equivalent(s)
ES electrospray
ES+ electrospray, positive ionization
Et ethyl
EtOAc ethyl acetate
EtOH ethanol
FC flash chromatography
h hour(s)
HOBt hydroxybenzotriazol
HPLC high performance liquid chromatography
LC-MS liquid chromatography-mass spectroscopy
Me methyl
MeOH methanol
min minute(s)
MS mass spectroscopy

### NCS N-chlorosuccinimide

org. organic
p para
PG protecting group
rt room temperature
sat. saturated
sol. solution
TBAC tetra-n-butylammonium chloride
TBME tert-butyl-methyl-ether
tBu tert-butyl
TFA trifluoroacetic acid
THF tetrahydrofuran
TLC thin layer chromatography
t_{R} retention time (in LC-MS or HPLC) given in minutes
UV ultra violet
Vis visible
xantphos 4,5-bis(diphenylphosphino)-9,9-dimethylxanthene
HPLC- or LC-MS-conditions (if not indicated otherwise):

Analytic: Zorbax 59 SB Aqua column, 4.6×50 mm from Agilent Technologies. Eluents: A: acetonitrile; B: H₂O+0.5% TFA. Gradient: 90% B→5% B over 2 min. Flow: 1 mL/min. Detection: UV/Vis+MS.

Preparative: Zorbax SB Aqua column, 20×500 mm from Agilent Technologies. Eluent: A: Acetonitrile; B: H₂O+0.05% ammonium hydroxide (25% aq.). Gradient: 80% B→10% B over 6 min. Flow: 40 mL/min. Detection: UV+MS, or UV+ELSD.

Chiral, analytic:
a) Regis Whelk column, 4.6×250 mm, 10 μm. Eluent A: EtOH+0.05% Et₃N.
Eluent B: hexane. Flow: 1 mL/min.
b) ChiralPak AD, 4.6×250 mm, 5 μm. Eluent A: EtOH+0.05% Et₃N. Eluent B: hexane. Flow: 1 mL/min.
c) ChiralCel OD, 4.6×250 mm, 10 μm. Eluent A: EtOH+0.1% Et₃N. Eluent B: hexane. Flow: 0.8 mL/min.
   Chiral, preparative:
a) Regis Whelk 01 column, 50×250 mm and a flow of 100 mL/min. Eluent A: EtOH+0.05% Et₃N. Eluent B: hexane.
b) ChiralCel OD, 20 μm, 50 mm×250 mm, flow 100 mL/min. Eluent A: EtOH+0.1% Et₃N. Eluent B: hexane.

### 5-Bromo-2-chloro-N-cyclopropylbenzamide

Into a flame-dried 250 mL round-bottom flask equipped with a magnetic stir bar and under N₂ were added 5-bromo-2-chlorobenzoic acid (10.0 g, 42.5 mmol) and DMF (3.9 mL, 51.0 mmol) in toluene (80 mL). The sol. was cooled to 0° C., and oxalyl chloride (4.4 mL, 51.0 mmol) was added dropwise over 1 h. The resulting mixture was stirred at 0° C. for 2 h and then the volatiles were removed. The resulting crude reaction mixture was dissolved in CH₂Cl₂ (100 mL) and cooled to 0° C. in an ice bath. Cyclopropylamine (4.5 mL, 63.7 mmol) was added dropwise over 1 h followed by addition of DIPEA (11.8 mL, 85.0 mmol). The resulting sol. was stirred at rt for 16 h. The reaction mixture was poured into a 1 L separatory funnel containing 1M aq. HCl (600 mL). The mixture was extracted with CH₂Cl₂ (6×250 mL). The combined org. layers were washed with brine, dried over MgSO₄, filtered and concentrated under reduced pressure. The product was crystallized from hexanes/CH₂Cl₂ and isolated by filtration to give the title compound (8.24 g, 71%).

### N-(5-bromo-2-chlorobenzyl)cyclopropylamine

A sol. of 5-bromo-2-chloro-N-cyclopropylbenzamide (12.0 g, 43.7 mmol) in THF (100 mL) was placed into a 250 mL round-bottom flask, equipped with a magnetic stir bar and under N₂. The sol. was treated with dropwise addition of BH₃.Me₂S (13.1 mL, 131 mmol), and the resulting suspension was stirred at rt for 1 h. The mixture was heated to reflux for 1 h, cooled to rt, and slowly quenched with dropwise addition of 1M aq. HCl (25 mL). The suspension was again refluxed for 1 h, cooled to rt, and basified to pH=10-11 with 1M aq. NaOH. The mixture was poured into a 500 mL separatory funnel containing 1M aq. NaOH (350 mL). The mixture was extracted with EtOAc (3×100 mL). The combined org. layers were washed with brine, dried over MgSO₄, filtered and concentrated under reduced pressure. The crude amine was used directly in the next step.

General Procedure for the Reductive Amination of Substituted Benzaldehydes with Cyclopropylamine:

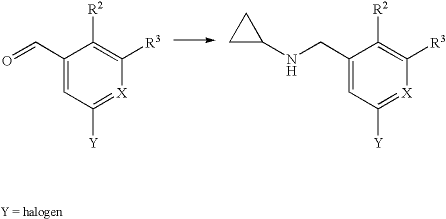

A sol. of substituted benzaldehyde (17.8 mmol, 1.0 eq.), cyclopropylamine (3.13 mL, 44.5 mmol, 2.5 eq.) and sodium cyanoborohydride (1.34 g, 21.4 mmol, 1.2 eq.) in MeOH (100 mL) was treated with dropwise addition of glacial AcOH (3.06 mL, 53.4 mmol, 3.0 eq.). The resulting sol. was stirred at rt for 16 h overnight. The reaction mixture was quenched with dropwise addition of sat. aq. NaHCO₃, and concentrated under reduced pressure to remove the MeOH. The crude residue was poured into a 250 mL separatory funnel containing sat. aq. NaHCO₃ (150 mL), and extracted with EtOAc (3×50 mL). The combined org. layers were washed with brine, dried over MgSO₄, filtered and concentrated under reduced pressure. Purification by FC yielded the benzamine product.

### General Procedure for the Boc-Protection of Cyclopropylbenzamines:

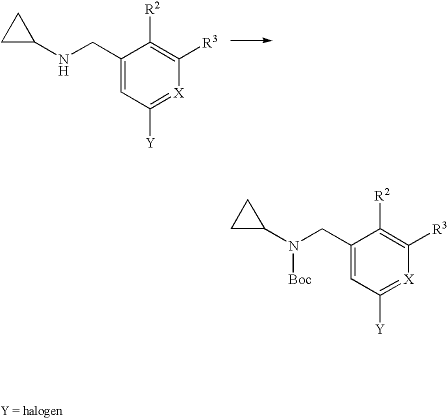

A sol. of the cyclopropylbenzamine (43.7 mmol, 1.0 eq.) in a biphasic mixture of CH₂Cl₂ (50 mL) and 1M aq. NaOH (50 mL) was treated with Boc₂O (15.1 mL, 65.6 mmol, 1.5 eq.). The mixture was stirred at rt vigorously for 16 h. The mixture was poured into a 500 mL separatory funnel containing H₂O (300 mL), and extracted with CH₂Cl₂ (3×100 mL). The combined org. layers were washed with brine, dried over MgSO₄, filtered and concentrated under reduced pressure. Purification by FC yielded the Boc-protected amine.

### General Procedure for the Allylation of Boc-Protected Cyclopropylbenzamines:

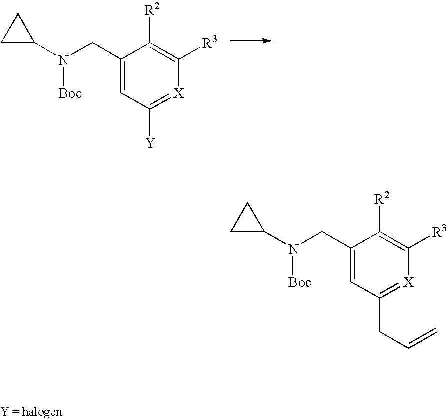

Into a flame-dried round-bottom flask or Schlenk tube, under N₂ was added Pd[PCy₃]₂ (0.05 eq.), CsF (2.0 eq.) and the corresponding aryl bromide (1.0 eq.). If the aryl chloride was being used as a starting material, the (Pd[PtBu₃]Br)₂ dimer (0.025 eq.) was used in place of the Pd[PCy₃]₂ catalyst. The flask was evacuated under reduced pressure (0.1 mm Hg) and backfilled with N₂ (repeated 3 times). The resulting solids were dissolved in anhydrous THF or dioxane (0.15 M sol.) and tri-n-butyl allyltin (1.5 eq.) was added and the resulting mixture was refluxed for 8-16 h, until TLC shows complete consumption of starting material. The reaction mixture was cooled to rt, and filtered through a pad of silica gel on a sintered glass funnel, washing with Et₂O. The filtrate was concentrated and purified by FC to give the corresponding allylbenzamide derivative.

### General Procedure for the Hydroboration/Oxidation of Allylbenzamines:

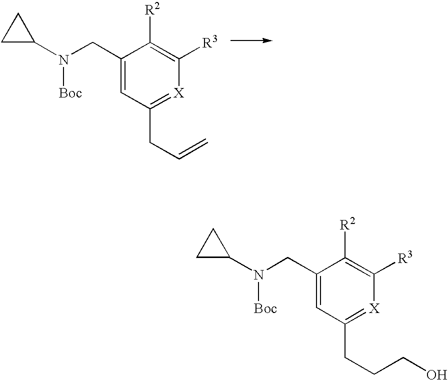

Into a flame-dried round-bottom flask equipped with a magnetic stir bar was added the allylbenzamine (1.0 eq.) and anhydrous THF (0.3 M sol.). The sol. was cooled to 0° C. and BH₃.Me₂S (1.1 eq.) was added dropwise over 20 min. The sol. was stirred at 0° C. for 1 h, then allowed to warm to rt, and stirred for an additional 2 h. The sol. was cooled to 0° C. and 1M aq. NaOH was added dropwise (CAUTION—EXOTHERMIC REACTION), followed by dropwise addition of 30% aq. H₂O₂. The mixture was allowed to warm to rt, and stirred for 2 h. The mixture was poured into a separatory funnel containing H₂O and extracted with Et₂O (3 times). The combined org. layers were washed with brine, dried over MgSO₄, filtered and concentrated under reduced pressure. Purification by FC yielded the desired alcohol product.

### General Procedure for the Oxidative Cleavage/Reduction of Allylbenzamines:

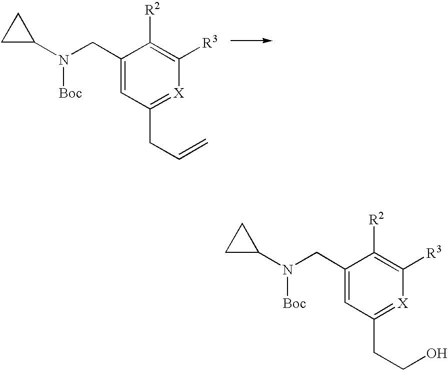

A sol. of allylbenzamine (1.0 eq.) in CH₂Cl₂ (0.4 M sol.) was cooled to −78° C. and O₃ gas was introduced into the sol. using a gas dispersion tube. The ozone gas was introduced until all of the starting material had been consumed, as determined by TLC, and the reaction mixture maintained a slight blue colour. The reaction was stirred at −78° C. for 20 min, then EtOH (0.5 M sol.) and NaBH₄ (2.5 eq.) were added. The mixture was allowed to warm to rt overnight (16 h). The reaction mixture was quenched with dropwise addition of sat. aq. NH₄Cl (5 mL), and poured into a separatory funnel containing sat. aq. NH₄Cl. The mixture was extracted with Et₂O (3 times). The combined org. layers were washed with brine, dried over MgSO₄, filtered and concentrated under reduced pressure. Purification by FC yielded the desired alcohol.

General Procedure for the Etherification of Aromatic Primary Alcohols with Methyl Iodide:

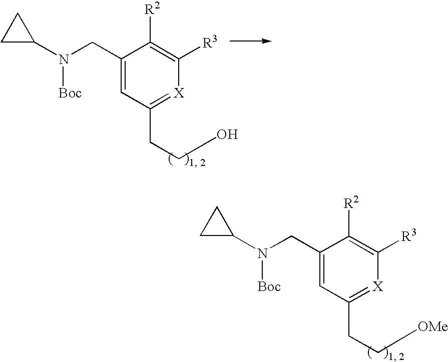

A suspension of the primary alcohol (1.0 eq.) in THF (0.25 M sol.) was cooled to 0° C. and treated with NaH (60% in oil, 2.0 eq.). The resulting mixture was stirred at 0° C. for 30 min and then at rt for another 30 min. The suspension was re-cooled to 0° C. and then MeI (8.0 eq.) was added in a single portion. The reaction mixture was stirred at 0° C. for 30 min, at rt for 30 min, and then heated to reflux for 4 h until all of the starting material was consumed as determined by TLC. The cooled reaction mixture was quenched with dropwise addition of sat. aq. NH₄Cl and poured into a separatory funnel containing sat. aq. NH₄Cl, and extracted with EtOAc (3 times). The combined org. layers were washed with brine, dried over MgSO₄, filtered and concentrated under reduced pressure. Purification by FC yielded the methyl ether.

### General Procedure for the Deprotection of Boc-Protected Cyclopropylbenzamines:

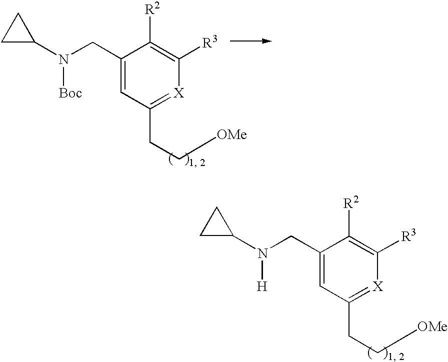

To a sol. of Boc-protected cyclopropylbenzamine (1.0 eq.) in CH₂Cl₂ (0.1-0.5 M sol.) was added 4 M HCl in dioxane (5.0 eq.). The resulting mixture was stirred at rt for 8-16 h until TLC shows complete conversion of starting material. The reaction was poured into a separatory funnel containing 1M aq. NaOH, and extracted with CH₂Cl₂ (3 times). Purification by FC yielded the corresponding free amine.

### 2-Bromo-5-chloro-pyridine-4-carbaldehyde

To a stirred sol. of diisopropylamine (20.9 mL, 148 mmol) in dry THF (350 mL) at −5° C. was added dropwise BuLi (1.6M in hexane, 89.5 mL, 143 mmol), and the resulting sol. was stirred for 30 min at −5° C. The sol. was allowed to cool to −70° C., and a sol. of 2-bromo-5-chloropyridine (25.0 g, 130 mmol) in THF (100 mL) was added dropwise at −70° C. over 15 min such that the internal temperature did not exceed −65° C. The mixture was stirred at −70° C. for 30 min. DMF (10.52 mL, 136 mmol) was added dropwise over 20 min such that the internal temperature did not exceed −70° C. The orange mixture was stirred at −70° C. for 40 min. The mixture was allowed to warm up to rt, and was poured onto a mixture of water (200 mL) and aq. 1M NaOH (50 mL). The mixture was extracted with EtOAc (2×), and the combined org. extracts were washed back with aq. 1M NaOH (2×). The org. extracts were dried over MgSO₄, filtered, and the solvents were removed under reduced pressure. Purification of the crude by FC (EtOAc/heptane 1:9→1:8→1:6→1:4→1:2→1:1) yielded the title compound (21.55 g, 72%). LC-MS: t_{R}=0.74 min; ES+: 295.01.

### 2-Bromo-5-chloro-4-dimethoxymethyl-pyridine

To a sol. of 2-bromo-5-chloro-pyridine-4-carbaldehyde (43.9 g, 199 mmol) in MeOH (800 mL) were successively added at rt trimethyl orthoformate (65.3 mL, 597 mmol) and p-toluenesulfonic acid monohydrate (1.90 g, 10.0 mmol). This reaction mixture was then heated to reflux for 3 h. The mixture was allowed to cool to rt and was concentrated under reduced pressure. The residue was dissolved in CH₂Cl₂, and this mixture was washed with aq. 10% K₂CO₃. The org. layer was dried over MgSO₄, filtered, and the solvents were removed under reduced pressure. Drying under high vacuum yielded the title compound (51.7 g, 97%). LC-MS: t_{R}=0.92 min; ES+: 309.06.

### 5-Chloro-4-dimethoxymethyl-2-(3-methoxy-propyl)-pyridine

To a suspension of Mg (911 mg, 37.5 mmol) and of iodine (one crystal) in dry THF (30 mL) was added dropwise 5% of the total amount of 1-bromo-3-methoxypropane (4.59 g, 30.0 mmol). The mixture was heated to reflux with the help of a heat gun until the Grignard formation had started. The rest of the 1-bromo-3-methoxypropane was added slowly, while an exothermic reaction proceeded. After the end of the addition, the reaction mixture was stirred under reflux for 20 min, and was allowed to cool to rt. This Grignard sol. (1M in THF, 23.5 mL, 23.5 mmol) was added dropwise to a mixture of 2-bromo-5-chloro-4-dimethoxymethyl-pyridine (2.50 g, 9.38 mmol) and Ni(dppp)Cl₂ (495 mg, 0.938 mmol) in THF (50 mL) at 0° C. The reaction mixture was stirred at rt for 30 min, and was then heated to reflux for 2 h. The mixture was allowed to cool to rt, and was dissolved with EtOAc. This mixture was washed with aq. sat. NaHCO₃. The org. layer was dried over MgSO₄, filtered, and the solvents were removed under reduced pressure. Purification of the residue by FC (heptane→EtOAc/heptane 1:1) yielded the title compound (1.51 g, 62%). LC-MS: t_{R}=0.80 min; ES+: 260.15.

### 5-Chloro-2-(3-methoxy-propyl)-pyridine-4-carbaldehyde

5-Chloro-4-dimethoxymethyl-2-(3-methoxy-propyl)-pyridine (25.5 g, 98.2 mmol) was dissolved in aq. 1M HCl (500 mL), and the mixture was heated to 80° C. for 2 h. The mixture was allowed to cool to rt, and EtOAc was added. The mixture was cooled to 0° C., and was basified with aq. 2.5M NaOH until a pH=10 was reached. The layers were separated, and the org. layer was dried over MgSO₄, filtered, and concentrated under reduced pressure. Drying the residue under high vacuum yielded the crude title compound (98.1 mmol, 99%) that was used further without purification. LC-MS: t_{R}=0.62 min; ES+: 246.12.

### [5-Chloro-2-(3-methoxy-propyl)-pyridin-4-ylmethyl]-cyclopropyl-amine

A mixture of 5-chloro-2-(3-methoxy-propyl)-pyridine-4-carbaldehyde (21.0 g, 98.2 mmol) and cyclopropylamine (13.8 mL, 196 mmol) in MeOH (450 mL) was stirred at rt overnight. NaBH₄ (4.83 g, 128 mmol) was added at 0° C., and the mixture was stirred at rt overnight. Ice was added, and the mixture was concentrated under reduced pressure. The crude product was dissolved in EtOAc, and this mixture was washed with aq. 1M NaOH. The aq. layer was extracted back with EtOAc. The combined org. extracts were dried over MgSO₄, filtered, and the solvents were removed under reduced pressure. Purification of the crude by FC (EtOAc/heptane 1:5→1:4→1:3→1:1→3:1→EtOAc) yielded the title compound (11.8 g) and [5-chloro-2-(3-methoxy-propyl)-pyridin-4-ylmethylene]-cyclopropyl-amine (10.7 g). This unreacted imine was dissolved in MeOH (20 mL), and this sol. was cooled to 0° C. NaBH₄ (3.20 g, 84.6 mmol) was added, and the mixture was stirred at rt overnight. NaBH₄ (3.20 g, 84.6 mmol) was added again, and the mixture was stirred for 3 days. Ice was added to the reaction mixture, and the mixture was concentrated under reduced pressure. The crude product was dissolved in EtOAc and the resulting mixture was washed with aq. 1M NaOH. The aq. phase was extracted back with EtOAc. The combined org. extracts were dried over MgSO₄, filtered, and the solvents were removed under reduced pressure. Purification of the crude by FC (EtOAc/heptane 1:3→1:2→1:1→EtOAc) yielded the title compound (9.4 g). The fractions of the title compounds were mixed together (21.2 g, 85%). LC-MS: t_{R}=0.55 min; ES+: 296.16.

### 2-(4-Bromo-phenoxy)-ethanol

4-Bromphenol (1003 g, 0.58 mol) was dissolved in xylenes (220 mL). [1,3]Dioxolan-2-one (53.7 g, 0.61 mol) and imidazole (592 mg, 8.70 mmol) were added. The mixture was heated to 140° C. for 3 days. The mixture was allowed to cool to rt, and the solvents were removed under reduced pressure. Drying the residue under high vacuum yielded the title compound (130 g, quantitative). LC-MS: t_{R}=0.81 min.

### Methanesulfonic acid 2-(4-bromo-phenoxy)-ethyl ester

2-(4-Bromo-phenoxy)-ethanol (125 g, 0.576 mol) was dissolved in CH₂Cl₂ (650 mL), and the sol. was cooled to 0° C. Et₃N (110 mL, 0.864 mol), then mesyl chloride (67.1 mL, 0.864 mol) were dropped at such a speed that the temperature did not raise above 10° C. (about 60 min). The mixture was stirred at 0° C. for 1 h, then at rt overnight. The mixture was diluted with CH₂Cl₂, and washed with brine (2×). The aq. phase was extracted back with CH₂Cl₂. The combined org. extracts were dried over MgSO₄, filtered, and the solvents were removed under reduced pressure. Drying the residue under high vacuum yielded the raw title compound (174 g, quantitative yield) that was used further without purification. LC-MS: t_{R}=0.92 min.

### 1-[2-(4-Bromo-phenoxy)-ethoxy]-2,6-dichloro-4-methyl-benzene

K₂CO₃ (29.3 g, 212 mmol) was dissolved in water (162 mL). 1-Propanol (150 mL) was added. A sol. of 2,6-dichloro-p-cresol (25 g, 141 mmol) in 1-propanol (150 mL) was added. Methanesulfonic acid 2-(4-bromo-phenoxy)-ethyl ester (41.6 g, 141 mmol) was added. The mixture was stirred at 85° C. for 6 h. The heating oil bath was removed, and water (330 mL) was added dropwise when the internal temperature had reached 78° C. The beige suspension was allowed to cool to rt. The mixture was filtered, and the precipitate was washed with water. Drying the precipitate under high vacuum at 30° C. for 48 h yielded the title compound (43 g, 81%). LC-MS: t_{R}=1.15 min.

### 2-(2,6-Dichloro-4-methyl-phenoxy)-ethanol

In a three-necked flask equipped with a gas droplet counter and an efficient cooling system, a mixture of 2,6-dichloro-p-cresol (20.0 g, 113 mmol), [1,3]dioxolan-2-one (9.95 g, 113 mmol) and imidazole (115 mg, 1.70 mmol) was heated to 160° C. for 25 h. The mixture was allowed to cool to rt. Purification by FC (Et₂O/heptane 1:1) yielded the title compound (18.7 g, 75%). LC-MS: t_{R}=0.88 min.

### 5-Bromo-2-[2-(2,6-dichloro-4-methyl-phenoxy)-ethoxy]-pyridine

A sol. of 2-(2,6-dichloro-4-methyl-phenoxy)-ethanol (18.6 g, 84 mmol) in THF (360 mL) was cooled to 0° C. NaH (about 55% in oil, 6.60 g, about 153 mmol) was added in portions, and the mixture was stirred at rt for 30 min. A sol. of 2,5-dibrompyridine (18.0 g, 76.3 mmol) in THF (60 mL) was added dropwise, and the mixture was heated to reflux for 90 min. The mixture was allowed to cool to rt, and ice was added carefully. The solvents were partially removed under reduced pressure, and the residue was diluted with EtOAc. This mixture was washed with aq. sat. NH₄Cl. The aq. layer was extracted back with EtOAc (2×). The combined org. extracts were washed with brine, dried over MgSO₄, filtered, and the solvents were removed under reduced pressure. Purification of the crude by FC (EtOAc/heptane 3:97) yielded the title compound (22.7 g, 79%). LC-MS: t_{R}=1.13 min; ES+: 378.08.

### 2-Chloro-3,6-difluoro-benzaldehyde oxime

2-Chloro-3,6-difluoro-benzaldehyde (25.0 g, 142 mmol) was dissolved in CH₃CN (175 mL). To this sol. was added NaHCO₃ (35.7 g, 424 mmol), and the mixture was stirred vigorously for 5 min. Water (350 mL) was added, and the mixture was stirred for 10 min. NH₂OH.HCl (19.7 g, 283 mmol) and TBAC (1.97 g, 7.08 mmol) were added, and the reaction mixture was stirred at rt for 1 h. AcOH (20 mL) was added dropwise to pH 6-7. The mixture was extracted with Et₂O (3×). The combined org. extracts were washed with brine, dried over Na₂SO₄, filtered, and the solvents were removed under reduced pressure. Drying under high vacuum yielded the title compound (25.0 g, 92%). LC-MS: t_{R}=0.93 min.

### (S)-1-(5-Bromo-pyridin-2-yl)-pyrrolidin-3-ol

A mixture of 2,5-dibromopyridine (12.2 g, 51.5 mmol) and (S)-hydroxypyrrolidine (2.80 g, 32.1 mmol) in toluene (50 mL) was heated to reflux overnight. The mixture was allowed to cool to rt, and the solvents were removed under reduced pressure. The residue was dissolved with EtOAc (150 mL), and the mixture was washed with aq. 10% K₂CO₃. The org. layer was dried over MgSO₄, filtered, and the solvents were removed under reduced pressure. Purification of the residue by FC (heptane→heptane/EtOAc 1:2) yielded the title compound (3.62 g, 46%). LC-MS: t_{R}=0.48 min; ES+: 243.15.

### (R)-5-Bromo-2-[3-(2,6-dichloro-4-methyl-phenoxy)-pyrrolidin-1-yl]-pyridine

Azodicarboxylate dipiperidide (11.7 g, 45.4 mmol) was added to a sol. of (S)-1-(5-bromo-pyridin-2-yl)-pyrrolidin-3-ol (8.82 g, 36.3 mmol) and 2,6-dichloro-p-cresol (7.37 g, 40.0 mmol) in toluene (200 mL). The mixture was degassed with nitrogen for 5 min, and PBu₃ (85%, 15.8 mL, 46.2 mmol) was added. The mixture was heated rapidly to 100° C., and stirred at this temperature for 2 h. The mixture was allowed to cool to rt, and was diluted with heptane (200 mL). The mixture was filtered, and the filtrate was evaporated under reduced pressure. Purification of the residue by FC (EtOAc/heptane 1:7) yielded a crude title compound that was diluted with CH₂Cl₂. This mixture was washed with aq. 1M NaOH. The org. layer was dried over MgSO₄, filtered, and the solvents were removed under reduced pressure. Drying the residue under high vacuum yielded the pure title compound (13.5 g, 93%). LC-MS: t_{R}=0.92 min; ES+: 402.98.

### (rac.)-3-[Cyclopropyl-(2,3-dimethyl-benzyl)-carbamoyl]-4-oxo-piperidine-1-carboxylic acid tert-butyl ester (B1)

A sol. of 4-hydroxy-5,6-dihydro-2H-pyridine-1,3-dicarboxylic acid 1-tert-butyl ester 3-methyl ester (WO 2004/105738, 1.00 g, 3.89 mmol), cyclopropyl-(2,3-dimethyl-benzyl)-amine (681 mg, 3.89 mmol) and p-toluenesulfonic acid monohydrate (92.4 mg, 0.486 mmol) in anhydrous toluene (40 mL) was stirred at reflux overnight in a Dean-Stark trap equipped flask. The reaction mixture was allowed to cool to rt. EtOAc (120 mL) was added, and the resulting mixture was washed successively with aq. sat. NaHCO₃ (2×), aq. 1M HCl (1×), and finally with aq. sat. NaHCO₃ (1×). The org. layer was dried over MgSO₄, filtered, and the solvents were removed under reduced pressure. Purification of the residue by FC (heptane→heptane/EtOAc 50:50) yielded the title compound (566 mg, 36%). LC-MS: t_{R}=1.02 min; ES+: 401.02.

### (rac.)-3-{[2-Chloro-5-(2-methoxy-ethyl)-benzyl]-cyclopropyl-carbamoyl}-4-oxo-piperidine-1-carboxylic acid tert-butyl ester (B2)

A sol. of 4-hydroxy-5,6-dihydro-2H-pyridine-1,3-dicarboxylic acid 1-tert-butyl ester 3-methyl ester (WO 2004/105738, 4.83 g, 18.8 mmol), [2-chloro-5-(2-methoxy-ethyl)-benzyl]-cyclopropyl-amine (3.00 g, 12.5 mmol) and p-toluenesulfonic acid monohydrate (298 mg, 1.56 mmol) in anhydrous toluene (188 mL) was stirred at reflux (oil bath at 130° C.) for 24 h in a Dean-Stark trap equipped flask. The mixture was allowed to cool to rt and left over the week-end. EtOAc (100 mL) was added, and the resulting mixture was washed successively with aq. sat. NaHCO₃, aq. 1M HCl (2×), and with brine. The org. layer was dried over MgSO₄, filtered, and the solvents were removed under reduced pressure. Purification of the residue by FC (heptane→heptane/EtOAc 40:60) yielded the title compound (2.39 g, 41%). LC-MS: t_{R}=1.03 min; ES+: 465.43.

### (rac.)-3-{[5-Chloro-2-(3-methoxy-propyl)-pyridin-4-ylmethyl]-cyclopropyl-carbamoyl}-4-oxo-piperidine-1-carboxylic acid tert-butyl ester (B3)

A sol. of 4-hydroxy-5,6-dihydro-2H-pyridine-1,3-dicarboxylic acid 1-tert-butyl ester 3-methyl ester (WO 2004/105738, 2.00 g, 7.77 mmol), [5-chloro-2-(3-methoxy-propyl)-pyridin-4-ylmethyl]-cyclopropyl-amine (1.98 g, 7.77 mmol) and p-toluenesulfonic acid monohydrate (185 mg, 0.972 mmol) in anhydrous toluene (78 mL) was stirred at reflux overnight in a Dean-Stark trap equipped flask. 4-Hydroxy-5,6-dihydro-2H-pyridine-1,3-dicarboxylic acid 1-tert-butyl ester 3-methyl ester (500 mg, 1.94 mmol) was added, and the mixture was heated to reflux for 4 h. The mixture was allowed to cool to rt. EtOAc was added, and the mixture was washed with aq. sat. NaHCO₃, aq. 1M HCl and aq. sat. NaHCO₃. The org. layer was dried over MgSO₄, filtered, and the solvents were removed under reduced pressure. Purification of the crude by FC (EtOAc/heptane 7:3) yielded the title compound (1.70 g, 46%). LC-MS: t_{R}=0.90 min; ES+: 480.39.

### (rac.)-(3S*,4R*)-3-[Cyclopropyl-(2,3-dimethyl-benzyl)-carbamoyl]-4-{4-[2-(2,6-dichloro-4-methyl-phenoxy)-ethoxy]-phenyl}-4-hydroxy-piperidine-1-carboxylic acid tert-butyl ester (D1)

A sol. of 1-[2-(4-bromo-phenoxy)-ethoxy]-2,6-dichloro-4-methyl-benzene (537 mg, 1.43 mmol) in dry THF (15 mL) at −78° C. was treated with BuLi (1.6M in hexane, 0.428 mL, 1.56 mmol). After 30 min this sol. was cannulated on a sol. of compound B1 (520 mg, 1.30 mmol) in dry THF (15 mL) at −78° C. After 1 h, the mixture was poured in aq. sat. NH₄Cl, extracted with EtOAc (2×), dried over Na₂SO₄, filtered, and the solvents were removed under reduced pressure. Purification of the residue by FC (heptane→heptane/EtOAc 70:30) yielded the title compound (89 mg, 10%). LC-MS: t_{R}=1.23 min; ES+: 697.16.

### (rac.)-(3R*,4S*)-3-{[2-Chloro-5-(2-methoxy-ethyl)-benzyl]-cyclopropyl-carbamoyl}-4-{4-[2-(2,6-dichloro-4-methyl-phenoxy)-ethoxy]-phenyl}-4-hydroxy-piperidine-1-carboxylic acid tert-butyl ester (D2)

A sol. of 1-[2-(4-bromo-phenoxy)-ethoxy]-2,6-dichloro-4-methyl-benzene (4.04 g, 10.8 mmol) in THF (107 mL) at −78° C. was treated with BuLi (1.6M in hexane, 7.38 mL, 11.8 mmol). After 30 min, DMPU (2.85 mL, 23.7 mmol) was added, and the mixture was stirred for 5 min. A sol. of compound B2 (2.00 g, 4.30 mmol) in THF (14 mL) was added slowly. The mixture was stirred for 15 min at −78° C., and aq. sat. NH₄Cl (100 mL) was added. The mixture was allowed to warm up to rt, and the solvents were partially removed under reduced pressure. The aq. residue was diluted with aq. sat. NH₄Cl (50 mL), and the mixture was extracted with EtOAc (3×). The combined org. extracts were dried over MgSO₄, filtered, and the solvents were removed under reduced pressure. Purification of the crude by FC (heptane→EtOAc/heptane 40:60) yielded the title compound (380 mg, 12%). LC-MS: t_{R}=1.27 min; ES+: 763.22.

### (rac.)-(3R*,4S*)-3′-{[2-Chloro-5-(2-methoxy-ethyl)-benzyl]-cyclopropyl-carbamoyl}-6-[2-(2,6-dichloro-4-methyl-phenoxy)-ethoxy]-4′-hydroxy 3′,4′,5′,6′-tetrahydro-2′H-[3,4′]bipyridinyl-1′-carboxylic acid tert-butyl ester (D3)

Mg turnings (535 mg, 22.0 mmol) and anhydrous LiCl (848 mg, 20.0 mmol) were placed in a dried flask in an oil bath at 120° C. overnight under high vacuum. Once this was cooled under N₂, without opening the flask, THF (10 mL) was added. A sol. of iso-propyl chloride in THF (10 mL) was slowly added at rt, and the mixture was stirred for 12 h at rt. The resulting grey IM-sol. is ready to be used but should not be kept more than 24 h. 5-Bromo-2-[2-(2,6-dichloro-4-methyl-phenoxy)-ethoxy]-pyridine (3040 mg, 8.07 mmol) in dry THF (80.6 mL) was treated with the previously prepared Grignard sol. (1M, 8.48 mL, 8.48 mmol). The mixture was stirred for 4 h at rt. The iso-propyl Grignard sol. (1M, 8.00 mL, 8.00 mmol) was added again, and the mixture was stirred for 2 h. A sol. of compound B2 (1500 mg, 3.226 mmol) in dry THF (15 mL) was added, and the mixture was stirred at rt for 15 min. The mixture was poured onto aq. sat. NH₄Cl, and extracted with EtOAc. The combined org. extracts were dried over Na₂SO₄, filtered, and the solvents were removed under reduced pressure. Purification of the residue by FC (heptane→heptane/EtOAc 70:30) yielded the title compound (1.74 g, 71%). LC-MS: t_{R}=1.23 min; ES+: 764.49.

### (rac.)-(3R*,4S*)-6-[3-(2-Chloro-3,6-difluoro-phenyl)-isoxazol-5-ylmethoxy]-3′-{[2-chloro-5-(2-methoxy-ethyl)-benzyl]-cyclopropyl-carbamoyl}-4′-hydroxy-3′,4′,5′,6′-tetrahydro-2′H-[3,4′]bipyridinyl-1′-carboxylic acid tert-butyl ester (D4)

Mg turnings (535 mg, 22.0 mmol) and anhydrous LiCl (848 mg, 20.0 mmol) were placed in a dried flask in an oil bath at 120° C. overnight under high vacuum. Once this was cooled under N₂, without opening the flask, THF (10 mL) was added. A sol. of iso-propyl chloride in THF (10 mL) was slowly added at rt, and the mixture was stirred for 12 h at rt. The resulting grey 1M-sol. is ready to be used but should not be kept more than 24 h. A sol. of compound K1 (1.08 g, 2.69 mmol) in dry THF (27 mL) was treated at rt with the previously prepared Grignard sol. (1M, 3.76 mL, 3.76 mmol). The mixture was stirred at rt and the formation of the Grignard was checked every hour. After 5 h, a sol. of compound B2 (500 mg, 1.08 mmol) in dry THF (10 mL) was added, and the reaction was stirred at rt for 1 h. The mixture was poured onto aq. sat. NH₄Cl, and the mixture was extracted with EtOAc. The combined org. extracts were dried over Na₂SO₄, filtered, and the solvents were removed under reduced pressure. Purification of the residue by FC (heptane→EtOAc/heptane 30:70) afforded the title compound (275 mg, 33%). LC-MS: t_{R}=1.20 min; ES+: 787.64.

### Mixture of (3′R,4′S)-3′-{[2-Chloro-5-(2-methoxy-ethyl)-benzyl]-cyclopropyl-carbamoyl}-6-[(R)-3-(2,6-dichloro-4-methyl-phenoxy)-pyrrolidin-1-yl]-4′-hydroxy-3′,4′,5′,6′-tetrahydro-2′H-[3,4′]bipyridinyl-1′-carboxylic acid tert-butyl ester and (3′S,4′R)-3′-{[2-Chloro-5-(2-methoxy-ethyl)-benzyl]-cyclopropyl-carbamoyl}-6-[(R)-3-(2,6-dichloro-4-methyl-phenoxy)-pyrrolidin-1-yl]-4′-hydroxy-3′,4′,5′,6′-tetrahydro-2′H-[3,4′]bipyridinyl-1′-carboxylic acid tert-butyl ester (D5)

Mg turnings (535 mg, 22.0 mmol) and anhydrous LiCl (848 mg, 20.0 mmol) were placed in a dried flask in an oil bath at 120° C. overnight under high vacuum. Once this was cooled under N₂, without opening the flask, THF (10 mL) was added. A sol. of iso-propyl chloride in THF (10 mL) was slowly added at rt, and the mixture was stirred for 12 h at rt. The resulting grey IM-sol. is ready to be used but should not be kept more than 24 h. (R)-5-Bromo-2-[3-(2,6-dichloro-4-methyl-phenoxy)-pyrrolidin-1-yl]-pyridine (2.16 g, 5.38 mmol) in dry THF (61 mL) was treated at rt with the previously prepared Grignard sol. (1M, 8.47 mL, 8.47 mmol). The mixture was stirred at rt and the formation of the Grignard was checked every hour. After 8 h, a sol. of compound B2 (1.13 g, 2.42 mmol) in dry THF (11 mL) was added and the reaction was stirred at rt for 1 h. The mixture was poured onto aq. sat. NH₄Cl, and the mixture was extracted with EtOAc. The combined org. extracts were dried over Na₂SO₄, filtered, and the solvents were removed under reduced pressure. Purification of the residue by FC (heptane→EtOAc/heptane 30:70) afforded the title compounds mixture (1.29 g, 68%). LC-MS: t_{R}=1.03 min; ES+: 787.77.

### (rac.)-(3′R*,4′S*)-3′-{[5-Chloro-2-(3-methoxy-propyl)-pyridin-4-ylmethyl]-cyclopropyl-carbamoyl}-6-[2-(2,6-dichloro-4-methyl-phenoxy)-ethoxy]-4′-hydroxy-3′,4′,5′,6′-tetrahydro-2′H-[3,4′]bipyridinyl-1′-carboxylic acid tert-butyl ester (D6)

Mg turnings (535 mg, 22.0 mmol) and anhydrous LiCl (848 mg, 20.0 mmol) were placed in a dried flask in an oil bath at 120° C. overnight under high vacuum. Once this was cooled under N₂, without opening the flask, THF (10 mL) was added. A sol. of iso-propyl chloride in THF (10 mL) was slowly added at rt, and the mixture was stirred for 12 h at rt. The resulting grey 1M-sol. is ready to be used but should not be kept more than 24 h. 5-Bromo-2-[2-(2,6-dichloro-4-methyl-phenoxy)-ethoxy]-pyridine (2.02 g, 5.37 mmol) in dry THF (54 mL) was treated at rt with the previously prepared Grignard sol. (1M, 7.51 mL, 7.51 mmol). The mixture was stirred at rt and the formation of the Grignard was checked every hour. After 5 h, a sol. of compound B3 (1.03 g, 2.15 mmol) in dry THF (10 mL) was added and the reaction was stirred at rt for 1 h. The mixture was poured onto aq. sat. NH₄Cl, and the mixture was extracted with EtOAc. The combined org. extracts were dried over Na₂SO₄, filtered, and the solvents were removed under reduced pressure. Purification of the residue by FC (heptane→EtOAc/heptane 2:7) afforded the title compound (921 mg, 55%). LC-MS: t_{R}=1.19 min; ES+: 779.64.

### (rac.)-(3′R*,4′S*)-3′-{[5-Chloro-2-(3-methoxy-propyl)-1-oxy-pyridin-4-ylmethyl]-cyclopropyl-carbamoyl}-6-[2-(2,6-dichloro-4-methyl-phenoxy)-ethoxy]-4′-hydroxy-3′,4′,5′,6′-tetrahydro-2′H-[3,4′]bipyridinyl-1′-carboxylic acid tert-butyl ester (D7)

A sol. of compound D6 (46 mg, 0.603 mmol) in dry CH₂Cl₂ (6.00 mL) was treated at rt with 3-chloroperbenzoic acid (70%, 166 mg, 0.675 mmol), and the mixture was stirred at rt for 2 h. The mixture was poured onto aq. sat. NaHCO₃, and extracted with EtOAc. The org. extract was washed with aq. sat. NaHCO₃ (2×), was dried over Na₂SO₄, filtered, and the solvents were removed under reduced pressure. Purification of the residue by FC (heptane→heptane/EtOAc 50:50) yielded the title compound (347 mg, 73%).

### 5-Bromo-2-[3-(2-chloro-3,6-difluoro-phenyl)-isoxazol-5-ylmethoxy]-pyridine (K1)

2,5-Dibromopyridine (31.4 g, 132 mmol) and compound L1 (25.0 g, 102 mmol) were dissolved in dry toluene (1.00 L) under nitrogen. tert-BuONa (14.7 g, 153 mmol), xantphos (3.54 g, 6.12 mmol) and Pd₂(dba)₃.CHCl₃ (1.83 g, 2.00 mmol) were added to the mixture. The mixture was heated to reflux overnight, and was allowed to was cool to rt. The mixture was washed with aq. sat. NaHCO₃ and brine. The org. layer was dried over MgSO₄, filtered, and the solvents were removed under reduced pressure. Purification of the residue by FC (EtOAc/heptane 10:90) yielded the title compound (17.4 g, 43%). LC-MS: t_{R}=1.08 min.

### [3-(2-Chloro-3,6-difluoro-phenyl)-isoxazol-5-yl]-methanol (L1)

A sol. of 2-chloro-3,6-difluoro-benzaldehyde oxime (21.3 g, 111 mmol) in DMF (66.7 mL) was added dropwise to a sol. of NCS (14.9 g, 111 mmol) and pyridine (1.78 mL) in DMF (222 mL). The mixture was stirred for 1 h at rt, and a sol. of propargyl alcohol (4.99 g, 89.1 mmol) in DMF (71 mL) was added dropwise. The reaction mixture was heated to 85° C., and a sol. of Et₃N (15.5 mL, 111 mmol) in DMF (89.3 mL) was slowly added. The reaction mixture was stirred at 85° C. for 60 min, and was allowed to cool to rt. The mixture was diluted with water (533 mL), and was extracted with EtOAc (2×). The combined org. extracts were washed with water and brine, were dried over Na₂SO₄, filtered, and the solvents were removed under reduced pressure. Purification of the residue by FC (EtOAc/heptane 40:60) yielded the title compound (17.0 g, 78%). LC-MS: t_{R}=0.84 min; ES+: 287.12.

### 3-(Benzyl-tert-butoxycarbonyl-amino)-propionic acid ethyl ester (M1)

Boc₂O (5.53 g, 25.3 mmol) was added to a sol. of N-benzyl-p-alanine ethyl ester (3.40 mL, 16.9 mmol) and DIPEA (11.6 mL, 67.6 mmol) in CH₂Cl₂ (200 mL) at 0° C. The mixture was stirred overnight while warming up to rt. The mixture was cooled to 0° C., and was partitioned with aq. 1M HCl. The org. layer was washed again with aq. 1M HCl and with aq. sat. NaHCO₃. The org. layer was dried over MgSO₄, filtered, and the solvents were removed under reduced pressure. Purification of the crude by FC (EtOAc/heptane 3:20) yielded the title compound (5.16 g, 99%). LC-MS: t_{R}=1.02 min.

### 3-(Benzyl-tert-butoxycarbonyl-amino)-propionic acid (N1)

A mixture of compound Ml (838 mg, 2.73 mmol) in EtOH (34 mL) and aq. 1M NaOH (13.7 mL) was stirred at 70° C. for 2 h. The mixture was allowed to cool to rt, and aq. 1M HCl was added until a pH=4 was reached. The solvents were partially removed under reduced pressure, and the aq. residue was extracted with EtOAc. The combined org. extracts were washed with brine, dried over MgSO₄, filtered, and the solvents were removed under reduced pressure. Drying under high vacuum yielded the crude title compound (769 mg, quantitative yield) that was used further without purification. LC-MS: t_{R}=0.89 min; ES+: 280.33.

### Benzyl-(2-{[2-chloro-5-(2-methoxy-ethyl)-benzyl]-cyclopropyl-carbamoyl}-ethyl)-carbamic acid tert-butyl ester (O1)

A mixture of compound N1 (769 mg, 2.75 mmol), DMAP (84.1 mg, 0.688 mmol), HOBt (446 mg, 3.30 mmol), DIPEA (1.78 g, 2.36 mmol) and EDC.HCl (1.32 g, 6.88 mmol) in CH₂Cl₂ (65 mL) was stirred at rt for 45 min. [2-Chloro-5-(2-methoxy-ethyl)-benzyl]-cyclopropyl-amine (1.14 g, 4.13 mmol) was added, and the mixture was stirred overnight. CH₂Cl₂ was added, and the mixture was washed with aq. 1M HCl (2×). The org. layer was dried over MgSO₄, filtered, and the solvents were removed under reduced pressure. Purification of the crude by FC (MeOH/CH₂Cl₂ 1:99) yielded the title compound (1.12 g, 76%). LC-MS: t_{R}=1.11 min; ES+: 501.30.

### 4-[2-(2,6-Dichloro-4-methyl-phenoxy)-ethoxy]-benzaldehyde (Q1)

BuLi (1.6M in hexane, 17.0 mL, 26.9 mmol) was added to a sol. 1-[2-(4-bromo-phenoxy)-ethoxy]-2,6-dichloro-4-methyl-benzene (8.81 g, 23.4 mmol) in THF (91 mL) at −78° C. The mixture was stirred for 10 min at −78° C., and DMF (2.72 mL, 35.1 mmol) was added. The mixture was stirred at −78° C. for 2.5 h, and aq. sat. NH₄Cl was added. The mixture was allowed to warm up to rt, and was extracted with TBME (2×). The combined org. extracts were washed with brine, dried over MgSO₄, filtered, and the solvents were removed under reduced pressure. Purification of the crude by FC (EtOAc/heptane 1:4) yielded the title compound (3.64 g, 48%). LC-MS: t_{R}=1.07 min; ES+: 325.03.

### EXAMPLES

### Example 1

### (rac.)-(3S*,4R*)-4-{4-[2-(2,6-Dichloro-4-methyl-phenoxy)-ethoxy]-phenyl}-4-hydroxy-piperidine-3-carboxylic acid cyclopropyl-(2,3-dimethyl-benzyl)-amide

A sol. of compound D1 (51 mg, 0.073 mmol) in dioxane (1 mL) at 0° C. was treated with HCl (4M in dioxane, 0.5 mL), and the mixture was stirred at 0° C. for 2 h. The reaction mixture was concentrated to dryness. Purification by FC(CH₂Cl₂→CH₂Cl₂/MeOH 90:10) yielded the title compound (18 mg, 39%). LC-MS: t_{R}=0.96 min; ES+: 597.16.

### Example 2

### (3S,4R)-4-{4-[2-(2,6-Dichloro-4-methyl-phenoxy)-ethoxy]-phenyl}-4-hydroxy-piperidine-3-carboxylic acid [2-chloro-5-(2-methoxy-ethyl)-benzyl]-cyclopropyl-amide

HCl (4M in dioxane, 2.40 mL) was added to a sol. of compound D2 (380 mg, 0.499 mmol) in CH₂Cl₂ (2.40 mL) at 0° C. The mixture was stirred for 2 h while warming up to rt, and the solvents were then removed under reduced pressure. Purification of the crude by FC (CH₂Cl₂/MeOH 90:10) yielded the racemic title compound (249 mg, 75%). This mixture was separated by chiral, preparative HPLC (Regis Whelk, isocratic eluent B 85%). The title compound was obtained (42 mg, 19%). LC-MS: t_{R}=0.96 min; ES+: 663.56. Chiral, analytic HPLC (Regis Whelk, isocratic eluent B 85%): t_{R}=33.0 min.

### Example 3

### (3′S,4′R)-6-[2-(2,6-Dichloro-4-methyl-phenoxy)-ethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide

Compound D3 (1.132 g, 1.485 mmol) was dissolved in CH₂Cl₂ (7.40 mL). The sol. was cooled to 0° C. HCl (4M in dioxane, 7.40 mL) was added dropwise to the mixture. The mixture was stirred for 1 h at rt, and was carefully poured onto a mixture of aq. sat. NaHCO₃ and EtOAc. The mixture was extracted with EtOAc. The combined org. extracts were dried over Na₂SO₄, filtered, and the solvents were removed under reduced pressure. Purification of the crude by FC(CH₂Cl₂ to CH₂Cl₂/MeOH 90:10) yielded the racemic title compound still mixed with little silica gel. This mixture was diluted with CH₂Cl₂, and filtered over cotton. The solvents were removed under reduced pressure to yield the pure, racemic title compound (904 mg, 92%). This racemate was separated by chiral, analytic HPLC (Chiralpack AD, isocratic eluent B 45%). The title compound was obtained (350 mg, 42%). LC-MS: t_{R}=0.94 min; ES+: 662.43. Chiral, analytic HPLC (Chiralpack AD, isocratic eluent B 65%): t_{R}=11.4 min.

### Example 4

### (3′S,4′R)-6-[3-(2-Chloro-3,6-difluoro-phenyl)-isoxazol-5-ylmethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide

Compound D4 (275 mg, 0.349 mmol) was dissolved in CH₂Cl₂ (1.75 mL). The sol. was cooled to 0° C. HCl (4M in dioxane, 1.75 mL) was added dropwise to the mixture. The mixture was stirred for 1 h at rt, and was carefully poured onto a mixture of aq. sat. NaHCO₃ and EtOAc. The mixture was extracted with EtOAc. The combined org. extracts were dried over Na₂SO₄, filtered, and the solvents were removed under reduced pressure. Purification of the crude by FC(CH₂Cl₂→CH₂Cl₂/MeOH 90:10) yielded the racemic title compound (162 mg, 67%). This racemate was separated by chiral, analytic HPLC (Chiralpack AD, isocratic eluent B 50%). The title compound was obtained (45 mg, 30%). LC-MS: t_{R}=0.92 min; ES+: 687.63. Chiral, analytic HPLC (Chiralpack AD, isocratic eluent B 50%): t_{R}=11.5 min.

### Example 5

### (3′S,4′R)-6-[(R)-3-(2,6-Dichloro-4-methyl-phenoxy)-pyrrolidin-1-yl]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide

Compounds D5 (1.29 g, 1.64 mmol) were dissolved in CH₂Cl₂ (8.2 mL). The sol. was cooled to 0° C. HCl (4M in dioxane, 8.2 mL) was added dropwise to the mixture. The mixture was stirred for 1 h at rt, and was carefully poured onto a mixture of aq. sat. NaHCO₃ and EtOAc. The mixture was extracted with EtOAc. The combined org. extracts were dried over Na₂SO₄, filtered, and the solvents were removed under reduced pressure. Purification of the crude by FC(CH₂Cl₂ to CH₂Cl₂/MeOH 90:10) yielded the title compound still mixed with its corresponding stereoisomer and with little silica gel. This mixture was diluted with CH₂Cl₂, and filtered over cotton. The solvents were removed under reduced pressure to yield the pure title compound mixed with its corresponding diastereoisomer (904 mg, 80%). Part of this mixture (150 mg) was separated by chiral, analytic HPLC (Chiralpack AD, isocratic eluent B 50%). The title compound was obtained (50 mg, 33%). LC-MS: t_{R}=0.81 min; ES+: 689.66. Chiral, analytic HPLC (Chiralpack AD, isocratic eluent B 50%): t_{R}=10.7 min.

### Example 6

### (3′S,4′R)-6-[2-(2,6-Dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-pyridin-4-ylmethyl]-cyclopropyl-amide

Compound D6 (920 mg, 1.18 mmol) was dissolved in CH₂Cl₂ (5.9 mL). The sol. was cooled to 0° C. HCl (4M in dioxane, 5.9 mL) was added dropwise to the mixture. The mixture was stirred for 1 h at rt, and was carefully poured onto a mixture of aq. sat. NaHCO₃ and EtOAc. The mixture was extracted with EtOAc. The combined org. extracts were dried over Na₂SO₄, filtered, and the solvents were removed under reduced pressure. Purification of the crude by FC(CH₂Cl₂ to CH₂Cl₂/MeOH 90:10) yielded the title compound still mixed its corresponding stereoisomer and with little silica gel. This mixture was diluted with CH₂Cl₂, and filtered over cotton. The solvents were removed under reduced pressure to yield the pure title compound mixed with its corresponding diastereoisomer (682 mg, 85%). Part of this mixture (80 mg) was separated by chiral, analytic HPLC (Chiralpack AD, isocratic eluent B 50%). The title compound was obtained (31 mg, 39%). LC-MS: t_{R}=0.88 min; ES+: 679.23. Chiral, analytic HPLC (Chiralpack AD, isocratic eluent B 50%): t_{R}=16.4 min.

### Example 7

### (3′S,4′R)-6-[2-(2,6-Dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-1-oxy-pyridin-4-ylmethyl]-cyclopropyl-amide

Compound D7 (347 mg, 0.500 mmol) was dissolved in CH₂Cl₂ (2.5 mL). HCl (4M in dioxane, 2.50 mL) was added dropwise to the sol. The mixture was stirred at rt for 1 h, and was carefully poured onto a mixture of aq. sat. NaHCO₃ and EtOAc. The mixture was extracted with EtOAc, dried over Na₂SO₄, filtered, and the solvents were removed under reduced pressure. Purification of the crude by FC(CH₂Cl₂→CH₂Cl₂/MeOH 9:1) yielded the title compound still mixed with silica gel. This mixture was taken in CH₂Cl₂, and filtered over cotton, which yielded the racemic title compound (266 mg, 77%). Part of this racemate (83 mg) was separated by chiral, analytic HPLC (Chiralpack AD, isocratic eluent B 50%). The title compound was obtained (29 mg, 35%). Chiral, analytic HPLC (Chiralpack AD, isocratic eluent B 50%): t_{R}=31.1 min.

### Biological Assays

### 1. Enzyme Immuno Assay (EIA) to Estimate AngI Accumulation and Renin Inhibition

### 1.1 Preparation of AngI-BSA Conjugate

1.3 mg (1 μmol) of AngI [1-10 (Bachem, H-1680)] and 17 mg (0.26 μmol) of BSA (Fluka, 05475) were dissolved in 4 mL of 0.1M phosphate buffer, pH 7.4, after which 2 mL of a 1:100 dilution of glutaraldehyde in H₂O (Sigma G-5882) was added dropwise. The mixture was incubated overnight at 4° C., then dialyzed against 2 liters of 0.9% NaCl, twice for 4 h at rt, followed by dialysis against 2 liters of PBS 1× overnight at rt. The solution was then filtered with a Syringe filter, 0.45 μm (Nalgene, Cat. No. 194-2545). The conjugate can be stored in polypropylene tubes in 0.05% sodium azide at 4° C. for at least 12 months.

### 1.2 Preparation of BSA-AngI Coated MTP

Microtiter plates (MPT384, MaxiSorp™, Nunc) were incubated overnight at 4° C. with 80 μl of AngI (1-10)/BSA conjugate, diluted 1:100'000 in PBS 1× in a teflon beaker (exact dilution dependent on batch of conjugate), emptied, filled with 90 μl of blocking solution [0.5% BSA (Sigma A-2153) in PBS 1×, 0.02% NaN₃], and incubated for at least 2 h at rt, or overnight at 4° C. 96 well MTP (MaxiSorp™, Nunc) were coated with 200 μl conjugate and blocked with 250 μl blocking solution as above, except that the blocking solution contained 3% BSA. The plates can be stored in blocking solution at 4° C. for 1 month.

### 1.3 AngI-EIA in 384 Well MTP

The AngI (1-10)/BSA coated MTP were washed 3 times with wash buffer (PBS 1×, 0.01% Tween 20) and filled with 75 μl of primary antibody solution (anti-AngI antiserum, pre-diluted 1:10 in horse serum), diluted to a final concentration of 1:100'000 in assay buffer (PBS 1×, 1 mM EDTA, 0.1% BSA, pH 7.4). 5 μl of the renin reaction (or standards in assay buffer) (see below) were added to the primary antibody solution and the plates were incubated overnight at 4° C. After the incubation the plates were washed 3 times with wash buffer and incubated with secondary antibody [anti-rabbit IgG, linked to horseradish peroxidase (Amersham Bioscience, NA 934V), diluted 1:2'000 in wash buffer] for 2 h at rt. The plates were washed 3 times with wash buffer and then incubated for 1 h at rt with substrate solution [1.89 mM ABTS (2.2′-azino-di-(3-ethyl-benzthiazolinsulfonate)] (Roche Diagnostics, 102 946) and 2.36 mM H₂O₂ [30%, (Fluka, 95300] in substrate buffer (0.1M sodium acetate, 0.05M sodium dihydrogen phosphate, pH 4.2). The OD of the plate was read at 405 nm in a microplate reader (FLUOStar Optima from BMG). The production of AngI during the renin reaction was quantified by comparing the OD of the sample with the OD of a standard curve of AngI (1-10), measured in parallel.

### 2. Primary Renin Inhibition Assay: IC₅₀ in Buffer, 384 Well MTP

The renin assay was adapted from an assay described before (Fischli W. et al., Hypertension, 1991, 18:22-31) and consists of two steps: in the first step, recombinant human renin is incubated with its substrate (commercial human tetradecapeptide renin substrate) to create the product Angiotensin I (AngI). In the second step, the accumulated AngI is measured by an immunological assay (enzyme immuno assay, EIA). The detailed description of this assay is found below. The EIA is very sensitive and well suited for renin activity measurements in buffer or in plasma. Due to the low concentration of renin used in this assay (2 fmol per assay tube or 10 pM) it is possible to measure inhibitor affinities in this primary assay down to low pM concentration.

### 2.1 Methodology

Recombinant human renin (3 pg/μl) in assay buffer (PBS 1×, 1 mM EDTA, 0.1% BSA, pH 7.4), human tetradecapeptide (1-14) substrate (Bachem, M-1120) [5 μM in 10 mM HCl], hydroxyquinoline sulfate (Fluka, 55100) [30 mM in H₂O] and assay buffer were premixed at 4° C. at a ratio of 100:30:10:145. 47.5 μl per well of this premix was transferred into polypropylene plates (MTP384, Nunc). Test compounds were dissolved and diluted in 100% DMSO and 2.5 μl added to the premix, then incubated at 37° C. for 3 h. At the end of the incubation period, 5 μl of the renin reaction (or standards in assay buffer) were transferred into EIA assays (as described above) and AngI produced by renin was quantified. The percentage of renin inhibition (AngI decrease) was calculated for each concentration of compound and the concentration of renin inhibition was determined that inhibited the enzyme activity by 50% (IC₅₀). The compounds exhibit a very good bioavailability and are metabolically more stable than prior art compounds.

### Examples of Inhibition:

| Compound of | |
|---|---|
| Example No. | IC₅₀ values [nM] |
| 1 | 0.18 |
| 3 | 0.12 |
| 5 | 0.15 |
| 7 | 0.23 |

1. A compound of the formula (I) 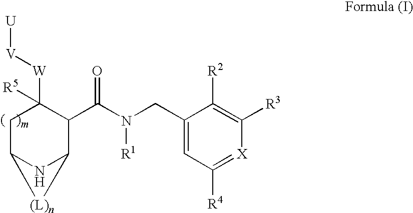
wherein
X represents CH, N, or N⁺—O⁻;
W represents a para-substituted phenyl, a para-substituted pyridinyl, or a thiazolyl;
V represents —CH₂CH₂CH₂—, —CH₂CH₂-A-, —CH₂-A-CH₂—, -A-CH₂CH₂—, —CH₂CH₂CH₂CH₂—, -A-CH₂CH₂CH₂—, —CH₂-A-CH₂CH₂—, —CH₂CH₂-A-CH₂—, —CH₂C₁₋₂CH₂-A-, -A-CH₂CH₂—B—, —CH₂CH₂CH₂CH₂CH₂—, -A-CH₂CH₂CH₂CH₂—, —CH₂-A-CH₂CH₂CH₂—, —CH₂CH₂-A-CH₂CH₂—, —CH₂CH₂CH₂-A-CH₂—, —CH₂CH₂CH₂CH₂-A-, -A-CH₂CH₂CH₂—B—, —CH₂-A-CH₂CH₂—B—, -A-CH₂CH₂—B—CH₂—, -A-CH₂CH₂CH₂—B—CH₂—, —CH₂-A-CH₂CH₂CH₂—B—, or —O—CH₂-Q-, wherein Q is bound to the group U of formula (I), or V represents a pyrrolidinyl of the formula: 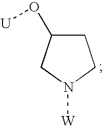
U represents unsubstituted aryl; mono-, di-, tri- or tetra-substituted aryl, wherein the substituents are independently selected from the group consisting of C₁₋₇-alkyl, —CF₃, halogen, and hydroxy-C₁₋₇-alkyl; or five-membered heteroaryl with two heteroatoms independently selected from nitrogen, oxygen and sulphur, wherein said heteroaryl radical is optionally mono-, di- or tri-substituted, wherein the substitutents are independently selected from the group consisting of C₁₋₇-alkyl, C₁₋₇-alkoxy, —CF₃, —OCF₃, and halogen;
Q represents a five-membered heteroaryl with two or three heteroatoms independently selected from O and N;
L represents —CH₂—CH₂—, —CH₂—CH(R⁶)—CH₂—, —CH₂—N(R⁷)—CH₂—, —CH₂—O—CH₂—, or —CH₂—S—CH₂—;
A and B represent independently from each others —O— or —S—;
R¹ represents C₁₋₇-alkyl or cycloalkyl;
R² represents halogen or C₁₋₇-alkyl;
R³ represents hydrogen, halogen, C₁₋₇-alkyl, C₁₋₇-alkoxy, or —CF₃;
R⁴ represents hydrogen; C₁₋₇-alkyl-O—(CH₂)₀₋₄—CH₂—; CF₃—O—(CH₂)₀₋₄-CH₂—; R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, C₁₋₄-alkoxy, —CF₃, —CH₂—CF₃, or cyclopropyl; or R¹³—C(═O)—(O)₀₋₁—(CH₂)₀₋₄—, wherein R¹³ is C₁₋₄-alkyl, C₁₋₄-alkoxy, or cyclopropyl; wherein R′ and R″ preferably do not both simultaneously represent hydrogen;
R⁵ represents hydroxy, C₁₋₇-alkoxy, hydroxy-C₁₋₇-alkyl, dihydroxy-C₁₋₇-alkyl, C₁₋₇-alkoxy-C₁₋₇-alkyl, C₁₋₇-alkoxy-C₁₋₇-alkoxy-C₁₋₇-alkyl, hydroxy-C₁₋₇-alkoxy-C₁₋₇-alkyl, carbamoyl-C₁₋₇-alkoxy, or C₁₋₇-alkyl-carbonyloxy;
R⁶ represents —H, —CH₂OR⁹, —CH₂NR⁸R⁹, —CH₂NR⁸COR⁹, —CH₂NR⁸SO₂R⁹, —CO₂R⁹, —CH₂OCONR⁸R⁹, —CONR⁸R⁹, —CH₂NR⁸CONR⁸R⁹, —CH₂SO₂NR⁸R⁹, —CH₂SR⁹, —CH₂SOR⁹, or —CH₂SO₂R⁹;
R⁷ represents —R⁹, —COR⁹, —COOR¹¹, —CONR⁸R⁹, —C(NR⁸)NR⁸′R⁹, —CSNR⁸R⁹, —SO₂R⁹, or —SO₂NR⁸R⁹; or R⁷ represents a radical of the formula: 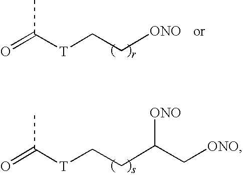
wherein T represents —CH₂—, —NH— or —O—, r is an integer from 1 to 6 and s is an integer from 1 to 4;
R⁸ and R⁸′ independently represent hydrogen, C₁₋₇-alkyl, C₂₋₇-alkenyl, cycloalkyl, or cycloalkyl-C₁₋₇-alkyl, wherein C₁₋₇-alkyl, cycloalkyl, and cycloalkyl-C₁₋₇-alkyl can be substituted by one, two, or three halogens;
R⁹ represents hydrogen, C₁₋₇-alkyl, cycloalkyl, or cycloalkyl-C₁₋₇-alkyl, wherein C₁₋₇-alkyl, cycloalkyl, and cycloalkyl-C₁₋₇-alkyl may be mono-, di- or tri-substituted, wherein the substituents are independently selected from the group consisting of halogen, hydroxy, —OCOR¹², —COOR¹², C₁₋₇-alkoxy, cyano, SO₂R¹², —CONR¹²R¹²′, morpholin-4-yl-CO—, ((4-C₁₋₇-alkyl)piperazin-1-yl)-CO—, —NHC(NH)NH₂, —NR¹⁰R¹⁰′ and C₁₋₇-alkyl, with the proviso that a carbon atom is attached at the most to one heteroatom in case this carbon atom is sp³-hybridized;
R¹⁰ and R¹⁰′ independently represent hydrogen, C₁₋₇-alkyl, cycloalkyl, cycloalkyl-C₁₋₇-alkyl, hydroxy-C₁₋₇-alkyl, —COOR⁸, or —CONH₂;
R¹¹ represents halogen, C₁₋₇-alkyl, C₁₋₇-alkoxy, —CF₃, or hydrogen;
R¹² and R¹²′ independently represent hydrogen, C₁₋₇-alkyl, C₂₋₇-alkenyl, cycloalkyl, or cycloalkyl-C₁₋₇-alkyl, wherein C₁₋₇-alkyl, cycloalkyl, and cycloalkyl-C₁₋₇-alkyl can be substituted by one, two, or three halogens;
n represents the integer 0 or 1; and
m represents the integer 0 or 1, with the proviso that m represents the integer 1 if n represents the integer 1;
and salts thereof.

2. A compound according to claim 1, wherein X represents N⁺—O⁻ and R⁴ represents C₁₋₄-alkoxy-C(═O)—NH—(CH₂)₀₋₄—CH₂— or R¹³—C(═O)—(O)₀₋₁—(CH₂)₀₋₄—, wherein R¹³ is C₁₋₄-alkyl, C₁₋₄-alkoxy, or cyclopropyl, or a salt of such a compound.

3. A compound according to claim 1, wherein X represents CH or N; and
R⁴ represents hydrogen; C₁₋₇-alkyl-O—(CH₂)₀₋₄—CH₂—; CF₃—O—(CH₂)₀₋₄—CH₂—; or R′₂N—(CH₂)₀₋₄—CH₂—, wherein R′ is independently selected from the group consisting of hydrogen, C₁₋₇-alkyl (optionally substituted by one to three fluorine), cyclopropyl (optionally substituted by one to three fluorine), cyclopropyl-C₁₋₇-alkyl (optionally substituted by one to three fluorine), and —C(═O)—R″ wherein R″ is C₁₋₄-alkyl, —CF₃, —CH₂—CF₃, or cyclopropyl;
or a salt of such a compound.

4. A compound according to claim 1, wherein X represents CH or N⁺—O⁻, or a salt of such a compound.

5. A compound according to claim 1, wherein R⁷ represents —R⁹, —COR⁹, —COOR¹¹, —CONR⁸R⁹, —C(NR⁸)NR⁸′R⁹, —CSNR⁸R⁹, —SO₂R⁹, or —SO₂NR⁸R⁹, or a salt of such a compound.

6. A compound according to claim 1, wherein A and B both represent —O—, or a salt of such a compound.

7. A compound according to claim 1, wherein R⁶ represents —CO₂CH₃ or —CO₂H, or a salt of such a compound.

8. A compound according to claim 1, wherein R⁷ represents —H, —COCH₃, —C(NH)NH₂, —CONHCH₂C(CH₃)₂CONH₂, —CONHCH(CH₂)₂, or —CONHC(CH₂)₂CN, or a salt of such a compound.

9. A compound according to claim 8, wherein R⁷ represents —H, or a salt of such a compound.

10. A compound according claim 1, wherein L represents —CH₂—CH₂— or —CH₂—NH—CH₂—, or a salt of such a compound.

11. A compound according to claim 1, wherein R¹ represents cyclopropyl, or a salt of such a compound.

12. A compound according to claim 1, wherein W represents a para-substituted phenyl, or 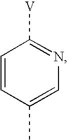
or a salt of such a compound.

13. A compound according to claim 1, wherein V represents —O—CH₂CH₂—O—, —O—CH₂-Q-, —CH₂—CH₂—O— wherein the —CH₂ part of —CH₂—CH₂—O— is bound to the group W of formula (I), or 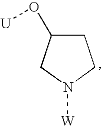
or a salt of such a compound.

14. A compound according to claim 13, wherein V represents —O—CH₂CH₂—O— or —O—CH₂-Q-, or a salt of such a compound.

15. A compound according to claim 1, wherein Q represents an isoxazolyl or an oxadiazolyl, or a salt of such a compound.

16. Compound according to claim 15, wherein Q represents an isoxazolyl, or a salt of such a compound.

17. A compound according to claim 1, wherein V-W represents: 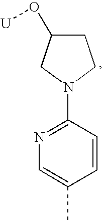
or a salt of such a compound.

18. A compound according to claim 1, wherein U represents: 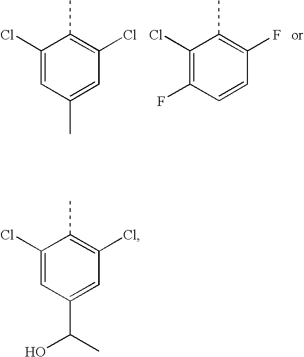
or a salt of such a compound.

19. A compound according to claim 18, wherein U represents: 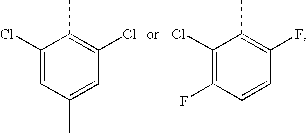
or a salt of such a compound.

20. A compound according to claim 1, wherein R² represents Cl, and R³ represents hydrogen, or a salt of such a compound.

21. A compound according to claim 1, wherein R⁴ represents CH₃—O—(CH₂)₂₋₃— or CH₃—C(═O)—NH—CH₂—CH₂—, or a salt of such a compound.

22. A compound according to claim 21, wherein R⁴ represents —CH₂CH₂CH₂—O—CH₃ or —CH₂CH₂—O—CH₃, or a salt of such a compound.

23. A compound according to claim 22, wherein R⁴ represents —CH₂CH₂—O—CH₃, or a salt of such a compound.

24. A compound according to claim 1, wherein R⁵ represents hydroxy, or a salt of such a compound.

25. A compound according to claim 1, wherein n represents the integer 0, or a salt of such a compound.

26. A compound according to claim 1, wherein the moiety 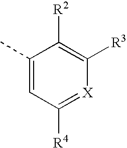
represents one of the following possibilities: 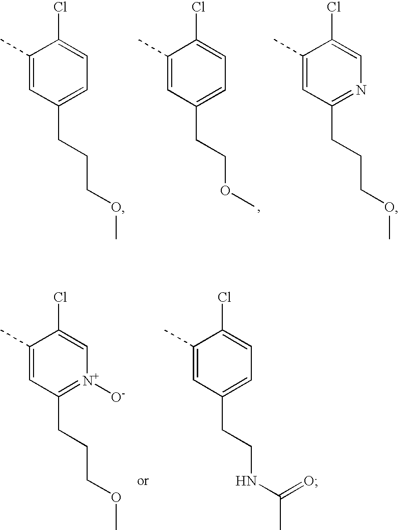
or a salt of such a compound.

27. A compound according to claim 1, wherein
X represents CH, N, or N⁺—O⁻;
W represents a para-substituted phenyl or a para-substituted pyridinyl;
V represents -A-CH₂CH₂—B— or —O—CH₂-Q-, wherein Q is bound to the group U of formula (I), or V represents a pyrrolidinyl of the formula: 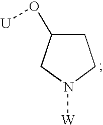
U represents tri-substituted phenyl, wherein the substituents are independently selected from the group consisting of C₁₋₇-alkyl and halogen;
Q represents an isoxazolyl;
A and B both represent —O—;
R¹ represents cyclopropyl;
R² represents halogen or C₁₋₇-alkyl;
R³ represents hydrogen or C₁₋₇-alkyl;
R⁴ represents C₁₋₇-alkyl-O—(CH₂)₀₋₄—CH₂—;
R⁵ represents hydroxy;
n represents the integer 0; and
m represents the integer 1,
or a salt of such a compound.

28. A compound according to claim 1, or a salt thereof, wherein the absolute configuration of a compound of formula (I) is as represented for formula (I′):

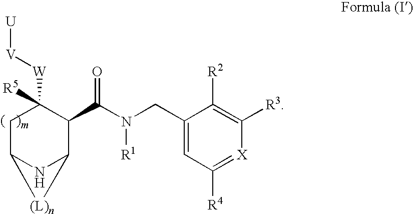

29. A compound according to claim 1, which is (3S*,4R*)-4-{4-[2-(2,6-dichloro-4-methyl-phenoxy)-ethoxy]-phenyl}-4-hydroxy-piperidine-3-carboxylic acid cyclopropyl-(2,3-dimethyl-benzyl)-amide, or a salt thereof.

30. A compound according to claim 1, selected from:
(3S,4R)-4-{4-[2-(2,6-dichloro-4-methyl-phenoxy)-ethoxy]-phenyl}-4-hydroxy-piperidine-3-carboxylic acid [2-chloro-5-(2-methoxy-ethyl)-benzyl]-cyclopropyl-amide,
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[3-(2-chloro-3,6-difluoro-phenyl)-isoxazol-5-ylmethoxy]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[(R)-3-(2,6-dichloro-4-methyl-phenoxy)-pyrrolidin-1-yl]-4′-hydroxy-1′,2′,3′,4′,5′,6′-hexahydro-[3,4′]bipyridinyl-3′-carboxylic acid [2-chloro-5-(2-methoxy-ethyl)-benzyl]-cyclopropyl-amide,
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-pyridin-4-ylmethyl]-cyclopropyl-amide, and
(3′S,4′R)-6-[2-(2,6-dichloro-4-methyl-phenoxy)-ethoxy]-4′-hydroxy-1′,2′,3′,4′,5′,6′-hexahydro-[3,4′]bipyridinyl-3′-carboxylic acid [5-chloro-2-(3-methoxy-propyl)-1-oxy-pyridin-4-ylmethyl]-cyclopropyl-amide,
or salts of such compounds.

31. A pharmaceutical composition comprising a compound according to claim 1, or a pharmaceutically acceptable salt thereof, and a pharmaceutically acceptable carrier material.

32. (canceled)

33. A method for treating a disease selected from hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases related to the renin-angiotensin system comprising administering to a patient in need thereof the composition of claim 1, or a pharmaceutically acceptable salt thereof.
